(19) United States
(12) Patent Application Publication
(10) Pub. No.: US 2026/0233026 A1
(43) Pub. Date: Aug. 13, 2026

(54) MULTI-SENSORY STIMULATION AND SENSOR FEEDBACK SYSTEM

(71) Applicant: Christopher Eric Neumann, Asheville, NC (US)

(72) Inventor: Christopher Eric Neumann, Asheville, NC (US)

(21) Appl. No.: 19/153,732

(22) PCT Filed: Feb. 5, 2024
(86) PCT No.: PCT/US24/14454

§ 371 Date: Aug. 5, 2025

Related U.S. Application Data
(60) Provisional application No. 63/483,446, filed on Feb. 6, 2023.

(51) Int. Cl. A61N 5/06; A61M 21/00; A61M 21/02

(52) U.S. Cl. CPC A61N 5/0622; A61M 21/02; A61M 2021/0044; A61M 2205/50; A61M 2205/8206; A61M 2205/8262; A61M 2230/005; A61M 2230/10; A61N 2005/0627; A61N 2005/0632; A61N 2005/0652; A61N 2005/0659; A61N 2005/0661

(57) ABSTRACT

The subject matter described herein includes methods and systems for a sensory stimulation feedback system. According to one embodiment, the sensory stimulation feedback system includes an outer unit having a first set of emitters arranged substantially in a same plane, and a modular inner unit including a second set of emitters arranged substantially in a same plane. A controller operates the emitters based on an input, which includes determining at least one of: a color, an intensity, a duration, a strobe effect, waveform type, duty cycle, and a strobe frequency of the emitters, where the emitters emit electromagnetic radiation having a wavelength between about 1500 nm (infrared) and about 100 nm (ultraviolet). A main body housing encloses an inner volume when coupled to the outer unit. The outer unit is coupled to the main body housing while the modular inner unit is detachably coupled to the outer unit.

### CROSS-REFERENCE TO RELATED APPLICATIONS

This application claims the benefit of priority of U.S. provisional patent application No. 63/483,446, titled “WELLNESS SENSORY STIMULATION SYSTEM UTILIZING ENERGY GENERATION MODULES CONTROLLED THROUGH A SENSOR DRIVEN FEEDBACK SYSTEM AND ASSOCIATED METHODS OF OPERATION”, filed on Feb. 6, 2023, which is incorporated by reference herein in its entirety.

### FIELD OF THE INVENTION

Embodiments of the present disclosure relate to sensory stimulation, and more particularly to a multi-sensory stimulation and sensor feedback system and associated method of operation.

### BACKGROUND

While light, encompassing both visible and invisible wavelengths, has traditionally been used for therapeutic and recreational purposes, existing technologies often lack the versatility to effectively manipulate and combine various sensory stimuli. Conventional light systems typically offer limited functionality, primarily providing unidirectional or static light patterns without the capacity for high-intensity, multi-color, and dynamically adjustable outputs. Similarly, existing auditory systems, such as conventional speakers or sound arrays, are often restricted in their ability to produce a range of calibrated sound frequencies essential for specific therapeutic or recreational experiences.

Furthermore, traditional sensory systems rarely incorporate tactile, olfactory, or gustatory stimuli effectively. Haptic devices, often used for tactile feedback, are usually limited in their integration with other sensory systems. Olfactory and gustatory stimulators, although beneficial for a comprehensive sensory experience, are seldom synchronized with optical or auditory stimuli in a cohesive manner.

Current sensory systems also tend to operate in isolation, lacking the capability to adapt dynamically to user responses or preferences. The integration of a sensor-driven feedback mechanism for real-time adaptation is generally absent, which limits the potential impact and effectiveness of these systems.

Therefore, there is a substantial need for a comprehensive sensory stimulation system that not only integrates multiple forms of stimuli, including light, sound, tactile, olfactory, and gustatory elements, but also incorporates a sophisticated feedback loop. Such a system should be capable of dynamically adjusting its output based on real-time user data, thus enhancing the overall sensory experience for wellness, recreational, or research purposes.

### SUMMARY

The present invention relates to a sensory stimulation system capable of inducing targeted biological reactions, including neurostimulation, by generating and controlling specific types of sensory stimuli through controlled energy generation, such as controlled light wavelengths, calibrated sound frequencies, and precise mechanical vibrations. The standard embodiment of the system is equipped with LED light sources, known for their energy efficiency and broad-spectrum capabilities. This choice reflects a commitment to an environmentally responsible and versatile multisensory experience. While LED lighting is the default energy emission, the system's modular design allows for easy adaptation to incorporate other lighting technologies or additional sensory modules as needed.

The system employs a range of interchangeable internal modular energy generation modules including, but not limited to, LED and halogen light sources for optical stimulation and speakers for auditory stimulation The system can also interface with external modules and systems such as haptic devices for tactile stimulation and with olfactory and gustatory stimulation systems, expanding its sensory range. The system is designed to easily interface, configure, and deliver these stimuli in a controlled manner, potentially influencing brain wave patterns, physical and emotional responses, and physiological states. While optimized to be used in a closed-loop feedback system, the invention is also designed for standalone operation, allowing for flexible use in various contexts without the need for continuous data feedback.

Users can interact with the system in multiple ways, including a user-friendly graphical user interface, mobile application, and more, allowing them to customize their experience according to personal preferences or to address specific wellness goals such as stress reduction, improved sleep quality, cognitive enhancement, all of which could increase a user's perception of their wellness.

The system incorporates a sensor-driven closed loop feedback mechanism, utilizing real-time data collection from user responses, such as sensor data (including EEG readings, heart rate monitor, etc.) or expressed preferences, to adapt the sensory output dynamically. The system employs machine learning models and advanced algorithms to learn and adapt to individual user patterns, enhancing personalization over time.

This invention finds potential applications in diverse fields including wellness settings for mood and cognitive disorders, immersive learning environments, and personalized entertainment systems. In addition to wellness and entertainment applications, the system serves as a valuable tool for research, providing researchers with a versatile platform for studying the effects of sensory stimulation on various biological and psychological parameters. The system's architecture allows for scalability, suitable for individual users or larger group environments like yoga studios, classrooms or research centers.

The system is designed with built-in safety protocols to prevent negative reactions to overstimulation, and its development adheres to relevant ethical guidelines and regulatory standards. The system is designed for seamless integration with existing ecosystems and is adaptable to future technological advancements in sensory stimulation and data analysis.

This system is intended for general wellness, personal development, and research purposes and is not designed to diagnose, treat, or manage any medical condition, ensuring its accessibility to a broad range of users. The system's design prioritizes user accessibility and ease of use, making advanced sensory stimulation technology available to the general public without specialized knowledge or training. The system ensures the highest standards of data security and privacy, with encrypted data storage and transmission protocols.

The subject matter described herein encompasses methods, systems, and computer program products for a comprehensive sensory stimulation feedback system. This system is not only adaptive to varying needs but also exhibits a wide range of embodiments due to its modular design.

In its core embodiment, the sensory stimulation feedback system comprises an enclosure, a processing module, and a power distribution module. This core setup facilitates the reception of input data from external sources and provides control signals to a diverse array of modular units. These units are not limited to optical emitters such as LED and halogen light sources, but also include modules for auditory stimulation (e.g., speakers), tactile stimulation (e.g., haptic devices), and interfaces for olfactory and gustatory stimulators, thereby providing a multisensory experience. Some units are specifically designed for active sensory stimulation, while others may serve aesthetic or supplementary roles.

A key aspect of the system is its user interaction capability. Users can engage with the system through various interfaces, including a graphical user interface or a mobile application, allowing for a highly personalized experience. This personalization is further enhanced by the system's ability to learn and adapt to individual user patterns over time, utilizing machine learning models and sensor data such as EEG readings and heart rate.

The processing module, equipped with a controller, operates the internal and external emitters and systems based on control data from various sources. For optical emitters, this involves determining parameters such as color, intensity, duration, strobe effect, waveform type, duty cycle, and strobe frequency, covering a wavelength range from about 1500 nm (infrared) to about 100 nm (ultraviolet). For non-optical emitters, the operation includes adjusting variables relevant to the specific sensory module, ensuring a cohesive multisensory output.

In addition to the controller's standard programming, the system includes methods for operating based on feedback from user-associated sensors, such as electroencephalographs. This allows the system to generate or modify output control signals in real-time, aligning with the user's brainwave patterns and sensory preferences.

Designed with scalability in mind, the system can be tailored for individual users or expanded for group settings like classrooms or wellness centers. It integrates seamlessly with existing ecosystems and is adaptable for future advancements in sensory technology. Prioritizing user safety and ethical standards, the system incorporates robust safety protocols and ensures the highest levels of data security and privacy.

### BRIEF DESCRIPTION OF DRAWINGS

FIG. 1 is a block diagram of a sensory stimulation system displaying the flow of operations according to an embodiment of the subject matter described herein.

FIG. 2 is a block diagram of the primary controller circuit of a sensory stimulation system, according to an embodiment of the subject matter described herein.

FIG. 3 is a block diagram of an emitter controller circuit of a sensory stimulation system, according to an embodiment of the subject matter described herein.

FIG. 4 is a block diagram of a seizure detection controller circuit of a sensory stimulation system, according to an embodiment of the subject matter described herein.

FIG. 5 is a block diagram of an external module interface controller circuit of a sensory stimulation system, according to an embodiment of the subject matter described herein.

FIG. 6 is a block diagram of a sensory stimulation system, according to an embodiment of the subject matter described herein.

FIG. 7 is a diagram of exemplary components of a sensory stimulation feedback system according to an embodiment of the subject matter described herein.

FIG. 8 is a flow chart illustrating exemplary steps of potential methods for controlling the operation of a sensory stimulation feedback system according to an embodiment of the subject matter described herein.

FIG. 9 is a diagram illustrating an exemplary configuration of a sensory stimulation feedback system for controlling two sets of emitters without using any input or control signals according to an embodiment of the subject matter described herein.

FIG. 10 is a diagram illustrating an exemplary configuration of a sensory stimulation feedback system for controlling two sets of emitters using one received control signal that is converted into two control signals according to an embodiment of the subject matter described herein.

FIG. 11 is a diagram illustrating an exemplary configuration of a sensory stimulation feedback system for controlling two sets of emitters with two input signals according to an embodiment of the subject matter described herein.

FIG. 12 is a diagram illustrating an exemplary configuration of a sensory stimulation feedback system for controlling two sets of emitters with two control signals according to an embodiment of the subject matter described herein.

FIG. 13 is a diagram illustrating an exemplary configuration of a sensory stimulation feedback system for controlling two sets of emitters with two input signals and two control signals according to an embodiment of the subject matter described herein.

FIG. 14 is a diagram illustrating an exemplary configuration of a sensory stimulation feedback system for controlling two sets of emitters with two input signals and two control signals according to an embodiment of the subject matter described herein.

FIG. 15 is an illustration of a dual zone (left side) and of a multi-zone (right side) sensory stimulation feedback system using a single source session control signal for dual axis (left side) and for multi-axis (right side) emission full spectrum entrainment according to an embodiment of the subject matter described herein.

FIG. 16 is an illustration of the front view of a sensory stimulation feedback system with a modular Halogen inner unit according to an embodiment of the subject matter described herein.

FIG. 17 is an illustration of the front view of a sensory stimulation feedback system with a modular high-density LED inner unit when powered on according to an embodiment of the subject matter described herein.

FIG. 18 is an illustration of the front view of a sensory stimulation feedback system with a modular high-density LED inner unit when powered off according to an embodiment of the subject matter described herein.

FIG. 19 is an illustration of the front view of a sensory stimulation feedback system without a modular inner unit according to an embodiment of the subject matter described herein.

FIG. 20 is an illustration of the rear view of a high-power LED light module of a sensory stimulation feedback system according to an embodiment of the subject matter described herein.

FIG. 21 is an illustration of the front view of a high-power LED light module of a sensory stimulation feedback system according to an embodiment of the subject matter described herein.

FIG. 22 is an illustration of the rear view of a sensory stimulation feedback system processing unit of a sensory stimulation feedback system according to an embodiment of the subject matter described herein.

FIG. 23 is an illustration of the front view of a sensory stimulation feedback system processing unit of a sensory stimulation feedback system according to an embodiment of the subject matter described herein.

FIG. 24 is an illustration of the rear view of a power distribution module of a sensory stimulation feedback system according to an embodiment of the subject matter described herein.

FIG. 25 is an illustration of the front view of a power distribution module of a sensory stimulation feedback system according to an embodiment of the subject matter described herein.

FIG. 26 is an illustration of the rear view of an outer housing of a sensory stimulation feedback system according to an embodiment of the subject matter described herein.

FIG. 27 is an illustration of the side view of an outer housing of a sensory stimulation feedback system according to an embodiment of the subject matter described herein.

FIG. 28 is an illustration of the rear view of a WS2813 addressable RGB LED module of a sensory stimulation feedback system according to an embodiment of the subject matter described herein.

FIG. 29 is an illustration of the front view of a WS2813 addressable RGB LED module of a sensory stimulation feedback system according to an embodiment of the subject matter described herein.

FIGS. 30A, 30B, and 30C are a schematic diagram of a DMX interface component of a sensory stimulation feedback system according to an embodiment of the subject matter described herein.

FIG. 31 is a schematic diagram of the ESP32 Integrated and RX/TX LED components of a sensory stimulation feedback system according to an embodiment of the subject matter described herein.

FIG. 32 is a schematic diagram of the power input, RGB indicator LED, Reset and boot button, and I2C pull up components of a sensory stimulation feedback system according to an embodiment of the subject matter described herein.

FIGS. 33A, 33B, and 33C are a schematic diagram of the voltage regulator, USB type C connector, and USB-to-UART components of a sensory stimulation feedback system according to an embodiment of the subject matter described herein.

FIGS. 34A, 34B, and 34C are a schematic diagram of an audio input component of a sensory stimulation feedback system according to an embodiment of the subject matter described herein.

FIGS. 35A, 35B, and 35C are a schematic diagram of the security SD card, cooling module connector, and accelerometer interface module components of a sensory stimulation feedback system according to an embodiment of the subject matter described herein.

FIGS. 36A, 36B, 36C, 36D, and 36E are a schematic diagram of the hex structure electrical contact components of a sensory stimulation feedback system according to an embodiment of the subject matter described herein.

FIGS. 37A and 37B are a schematic diagram of the modular inner unit connector components of a sensory stimulation feedback system according to an embodiment of the subject matter described herein.

FIG. 38 is an illustration of the front view of a modular high-density LED inner unit when powered off according to an embodiment of the subject matter described herein.

FIG. 39 is an illustration of the front view of a modular Halogen inner unit when powered off according to an embodiment of the subject matter described herein.

FIG. 40 is an illustration of the rear view of the universal modular inner unit connections according to an embodiment of the subject matter described herein.

FIG. 41 is an illustration of the front view of a sensory stimulation feedback system with installed LED lenses for the First Set of Emitter(s) [Subset A] and a diffusion layer without a modular inner unit according to an embodiment of the subject matter described herein.

FIG. 42 is an illustration of the rear view of a sensory stimulation feedback system with an installed cooling module according to an embodiment of the subject matter described herein.

FIG. 43 is a representation of seven separate sensory stimulation feedback systems aligned in a matrix where all of the controllers are configured in the same way and are all receiving the same control signal independently which results in all seven lights activating the same emitters, producing the same effect according to an embodiment of the subject matter described herein.

FIG. 44 is a representation of seven separate sensory stimulation feedback systems aligned in a matrix where all of the controllers are configured in the same way but are all receiving different control signals independently which results in all seven lights activating different individual emitters, producing a different effect on each device but creating a shared pattern within the matrix according to an embodiment of the subject matter described herein.

FIG. 45 is a representation of a dual theremin controller, designed to control specific mapped variables of a sensory stimulation feedback system according to an embodiment of the subject matter described herein.

FIGS. 46A and 46B is a representation of a graphical user interface, designed to control specific mapped variables of a sensory stimulation feedback system according to an embodiment of the subject matter described herein.

FIG. 47 is a representation of a light and sound hybrid layout of a sensory stimulation feedback system, including six full range speakers, one set of light emitters including the WS2813 layer ([subset B]) and six high powered RGBW LEDs (subset A), and a modular Halogen inner unit according to an embodiment of the subject matter described herein.

FIG. 48 is a representation of a light and sound hybrid layout of a sensory stimulation feedback system, including six full range speakers, one set of light emitters including the WS2813 layer ([subset B]) and six high powered RGBW LEDs (subset A), and a modular speaker inner unit according to an embodiment of the subject matter described herein.

FIG. 49 is a representation of an internal electrical routing distribution hexagon structure of a sensory stimulation feedback system. The six-sided hexagon structure is assembled to meet the requirements of the desired sensory stimulation feedback system configuration. FIG. 49 includes two blank faces, one Data Face, one AC Face, one High Density Power Face, and one WS2813 Power Face according to an embodiment of the subject matter described herein.

### DETAILED DESCRIPTION

The energy emission system described herein utilizes biofeedback and sensory stimulation. This energy emission system is designed to stimulate all five senses, utilizing advanced biofeedback mechanisms as a guide. By interpreting data from biological sensors, the system can tailor sensory experiences, potentially offering users a deeper insight into their own consciousness.

The system comprises an array of energy emitters, each meticulously engineered to interact with specific senses. For sound, high-fidelity auditory stimulators capable of producing a broad range of frequencies are employed. For sight, emitters range from the infrared to the ultraviolet spectrum, enabling an optical experience beyond the ordinary. Touch is engaged through haptic feedback mechanisms, while olfactory and gustatory experiences are evoked using advanced scent and taste emitters.

Thus, the present invention pertains to an advanced sensory stimulation system intricately designed for enhancing consciousness and promoting wellness. More specifically, it relates to a sophisticated, integrated system capable of generating, modulating, and controlling a wide spectrum of sensory stimuli through a network of internal and/or modular energy generation units, each fine-tuned and orchestrated based on real-time, adaptive feedback mechanisms. The system stands at the confluence of neurostimulation, multisensory integration, and personalized wellness, employing cutting-edge technologies to create immersive, transformative experiences. It leverages data analysis, machine learning models, and user-specific biofeedback to dynamically adjust sensory outputs, aiming to foster profound, positive shifts in consciousness and overall well-being.

The system transcends traditional neurostimulation technologies like TMS, tDCS, and DBS by providing a holistic wellness solution. It operates not just as a controller capable of neurostimulation but as a multisensory synchronized integrated ecosystem for calibrated sensory experiences based on real time data from a user. The system is adept at functioning autonomously, capable of operating as a standalone controller devoid of external emitters, or in conjunction with a suite of sensory modules and/or emitters. Its inherent embodiment incorporates energy emitters in the form of LED light sources for their broad spectrum and energy efficiency, but the modular architecture ensures that the system's capabilities are not confined to light experiences alone.

The system can adapt and evolve. Internal and external modular components—ranging from LEDs and unfiltered halogen bulbs for optical stimulation to speakers for auditory stimulation, from essential oil diffusers for olfactory experiences to haptic devices for tactile feedback as well as artificial taste emitters for gustatory stimulation which can be interchanged and combined, allowing the system to cater to an ever-changing extensive array of time synched sensory needs. In one embodiment, the system includes a three-dimensional hexagonal form factor that provides an aesthetic and functional structure for the integration of these components and also makes the system scalable and adaptable.

The system can interface as a closed-loop feedback mechanism and leverages real-time data—from EEG readings, heart rate monitors, and more—to dynamically adapt the sensory output based on the data provided. By incorporating advanced machine learning models, artificial intelligence algorithms, and databases, the system learns and evolves with each interaction, ensuring a personalized experience for each user.

By capturing, storing, and analyzing data from users, including physiological responses such as brainwave and heart rate changes, the system employs machine learning and artificial intelligence to refine its understanding of the effects of specific controlled sensory stimulations. This data-driven approach allows the system to tailor its output, enhancing the precision, ensure the ability to replicate experiences, and safely provide users a sensory stimulation experience that they can safely enjoy.

Cognitively, we interface with our physical external reality solely through the awareness and processing of our five primary senses. Our internal reality is directly influenced by the stimuli provided by our physical external reality, but our internal reality has no bearing on our external reality, other than how we choose to react to the stimuli provided to us.

Our perceptions and perspectives are a result of the natural stimulations of our five primary senses within this shared reality. Our knowledge, experiences, and memories are constantly reinforced by what and how we react with and to regarding the different stimulations and sensations within our personal experiences of reality.

Wellness, a subjective state, is not just a physical condition but also a psychological one, influenced significantly by sensory experiences. For instance, the calming effect of a forest's natural sounds or the invigorating impact of a vibrantly lit workspace. By artificially overstimulating one or more of our five primary senses, we are able to lessen our awareness of stressors.

Historically, cultures worldwide have used rhythmic drumming, incense, and visual patterns in rituals, intuitively leveraging sensory stimulation for therapeutic and spiritual purposes. From the simple kaleidoscopes of the 19th century to modern VR headsets, sensory technology has evolved to provide increasingly immersive and controllable experiences.

Through artificial stimulations such as binaural beats, stroboscopic lights, acupuncture, aroma therapy, and different tastes, we are able to artificially stimulate the senses of sound, sight, touch, smell, taste.

One example of artificial sensory stimulation is that artificial light, especially in specific wavelengths and/or patterns, can be used to create targeted emotional or cognitive responses, such as relaxation or heightened alertness.

Artificial stimulation could generate feelings of euphoria, bliss, peace, awe, wonder, confusion, or fear depending on a person's interpretation of the experience. The resulting perception of this stimulation may not be able to be described (accurately or at all) or understood by the individual experiencing the stimulation or to an observer.

As the brain processes sensations, the person's body and brain can be affected. These reactions can be observed with the use of sensors such as a heart rate monitor, an EEG (i.e. electroencephalograph), and others. By monitoring a user's reaction, modified reaction, or lack of reaction to artificial stimulation through the use of a sensor or a combination of different types of sensors (e.g., EEG, EMG, heart rate variability, thermometer, etc.) one can observe the actual effects of the stimulation on a user without biased interpretation. By relying solely on the recorded sensor data of the person being stimulated, one would be able to bypass a user's inability to accurately describe an experience as well as any inaccurate interpretations or confusion from the user.

The development of machine learning models introduced and paved the way for the potential to collect, analyze, and process large amounts of data. Combining various energy generation types to artificially manipulate sensory stimulation responses, sensors (including EEG, EMG, heart rate monitors, etc.), and real time (or close to real time) machine learning models to create a closed loop feedback system that can generate and control energy in specific ways to affect a user's biological experience that is controlled by a computer program which uses the user's real time sensor data to determine the ways in which to manipulate the energy could be revolutionary.

Additionally, by structuring algorithms based on sensor data, the variables pertaining to the types of stimulation (e.g., audio volume, light brightness, tactile pressure, olfactory sweetness, gustatory bitterness, etc.) could be modified and manipulated in order to achieve various sensory amplitudes thus resulting in various altered states.

It may be advantageous to produce a sensory stimulation feedback system that is capable of monitoring a user's biosensor data in real time (or close to real time) while simultaneously altering the variables associated with different types of artificial stimulation through the generation and control of different types of energy emitters.

Accordingly, a need exists for a device capable of receiving, storing, and analyzing data from external inputs, generating stimulation variables based on that data, and either generating or modifying control signals to internal or external systems and/or stimulation emitters (e.g., LED arrays, speakers, tactile transducers, olfactory systems, gustatory systems, etc.).

The subject matter described herein includes a sensory stimulation feedback system created to produce and/or trigger multiple types of sensory stimulations including auditory (through the manipulation of audio inputs), tactile (through the triggering of transducers or other tactile sensory experiences), gustatory (through the activation of an external system), olfactory (through the activation of an external oxygen bar for example), and optically (through various patterns of light between infrared and ultraviolet wavelengths that, when observed by a user, may have various effects on the brain activity or brainwaves of the user).

Input(s) to the sensory stimulation feedback system may include, among other sources, real-time and/or pre-recorded data from an EEG device associated with a user. This may allow users to experience a desired brainwave pattern by observing a light pattern associated with inducing the desired brainwave pattern or state.

In contrast to conventional configurations, which are only capable of producing simple patterns using a limited number and limited range of light sources, the present disclosure includes not only independently controlled light sources but a large array of varying types of sensory stimulation options as well as the ability to affect the variables associated with the various sensory stimulation options, including internally controlled light emitters. These light sources are capable of being individually controlled or divided into groups, or zones, for displaying multiple patterns simultaneously. This allows for not only a personalized experience based upon sensors recording and processing the user's brainwave and biological reactions but also a safer experience for the user, rather than what conventional systems have ever been able to offer.

In other words, the subject matter described herein includes (in this incarnation) a multi-axis, multi-optic, multi-channel, multi-chromatic multi-sensory entrainment system, emission synchronizer, recorder, analyzer, and playback optimization system designed to generate, synchronize, and modify energy wave patterns and energy and/or matter disbursement from one or multiple energy and matter emitting sources distributed in a modular wired and/or wireless circuit to produce dynamic harmonic and disharmonic wave interference patterns.

Brainwave entrainment, also referred to as “entrainment”, “brainwave synchronization”, or “neural entrainment”, refers to the natural synchronization of brainwaves (large-scale electrical oscillations in the brain) in response to the rhythm of periodic external stimuli, such as flickering lights, binaural beats, etc. Different states of consciousness may be associated with different dominant brainwave frequencies. As such, brainwave entrainment may be used to induce a desired state.

One example of a state of consciousness that may be recorded and/or reproduced according to the subject matter described herein includes “hypnagogia”. Hypnagogia is the experience of the transitional state from wakefulness to sleep and is characterized by stage 1 of NREM sleep, pre-sleep alpha waves, and drop-offs in alpha EEG activity. In a hypnagogic state, a user may experience hallucinations, lucid thought, exploding head syndrome, lucid dreaming, and sleep paralysis.

As every brain is different and every brain is constantly changing states, there is no way to find a “baseline” between users. Because of this, sensory stimuli can affect different people differently and can affect the same person differently day to day. For example, one light pattern may put someone into a deep state of hypnagogia one day, the next day, it may have an entirely different effect. Therefore historically, artificial sensory stimulations create completely different experiences for everyone, every time.

Much like consciousness and the human experience, the impact that light has on the human psyche, and the human experience, is only partially understood. Humans are literally beings made of light, as our DNA both emits and absorbs light. Light intensity drives activity and rest patterns built into the DNA of nearly all living species while the timing of light emission (which we interpret as colors on a targeted spectrum map) has notable and long-documented influence on a similar, yet less-documented range of human emotions. Understood is the duplicity of light and its ability to act as “bundles” of electromagnetic energy (photons) while simultaneously interacting with other photons in synchronized, wave-like patterns which can cancel, amplify, and/or create new patterns of intensities and frequencies at varying spaces and times (in relation to the source or sources of the energy being emitted).

It is appreciated, however, that while “light” in its varying states of energy and emission affects the human experience, the specific mapping of factors such as energy, time, and/or space relative to the sources of light, as well as the positioning of an individual experiencing such effects, may be observed, determined, and refined according to the methods and systems disclosed herein. Similarly, while there is data on electromagnetic activity in the human brain when exposed to real-world events or multimedia such as photos, audio and/or video, the data is limited when addressing the effects of energy dispersion and interaction in the form of more dynamic and complex light wave interference patterns found not in multimedia alone, yet found in abundance in the more chaotic natural world.

Finally, conventional combinations of light sources controlled by computational logic have fixed and/or limited physical arrangements in order to specifically induce a limited range of topical human experiences (such as “Restful” or “Hypnagogic” states of mind) and such devices are limited by a fixed positioning of their singular or minimally-paired emitter or emitters, and/or do not offer dynamic, granular, feedback, recording, and/or externally-sourced multimedia synchronization capabilities. As a result, such conventional light sources provide a limited range of static emission capabilities, little to no efficacy data, and little to no automated, reproducible experimentation capabilities required to better understand the complexities of the spectrum of effects such emitters have on the human experience.

It is therefore appreciated that a device or system capable of capturing an individual's baseline brain wave state, projecting a multitude of synchronized energy from a modular, dynamic energy emitter array, that results in a wide range of dynamic, yet reproducible wave interference patterns in the environment, as well as behind the optic sensors of an individual experiencing the effect, would be desirable. Also appreciated is that specific sequences of documented energy emission and confluence patterns, produced in isolation or in the presence of synchronized multimedia, and recorded alongside other human experience data collected in association with these patterns for the purpose of analysis and/or targeted playback would be beneficial.

As will be described in greater detail below with respect to the Figures, a multi-axis, multi-optic, multi-channel, multi-chromatic multi-sensory entrainment system, emission synchronizer, recorder, analyzer, and playback design is provided. The multi-axis, multi-optic, multi-channel, multi-chromatic multi-sensory entrainment system, emission synchronizer, recorder, analyzer, and playback device can operate with one, two, or a multitude of modular energy sources and/or systems working independently, as mixed sets, and/or as a singular energy emission source to create, record, and play back targeted energy emission sequences in sync with externally-sourced multimedia and/or documented human interaction data.

The present invention will now be described more fully hereinafter with reference to the accompanying figures in which exemplary embodiments of the invention are shown. However, the invention may be embodied in many different forms and should not be construed as limited to the representative embodiments set forth herein. The exemplary embodiments are provided so that this disclosure will be both thorough and complete and will fully convey the scope of the invention and enable one of ordinary skill in the art to make, use, and practice the invention.

Relative terms such as lower or bottom; upper or top; upward, outward, or downward; forward or backward; and vertical or horizontal may be used herein to describe one element's relationship to another element illustrated in the figures. It will be understood that relative terms are intended to encompass different orientations in addition to the orientation depicted in the drawings.

In a core embodiment, a sensory stimulation feedback system consists of an enclosure, a processing module, a power distribution module, and an internal electrical routing distribution hexagon structure. In addition to housing and supplying power to the various units, the core embodiment is capable of receiving input data from internal and/or external sources and providing control signals to modular inner units, outer units, and to other connected devices and systems. Some units include emitters such as light emitters and sound emitters while others may be blank or purely decorative.

The enclosure houses the internal components including the processing module, the power distribution module, the internal electrical routing distribution hexagon, and a variety of different types of interchangeable modular inner and outer units. The enclosure is designed to securely mount the internal and external modules and components, accommodate airflow within the enclosure, provide a durable protective environment for the internal components, allow for the attachment of additional accessories, components, and modular units while supplying the needed space for accommodating external connections.

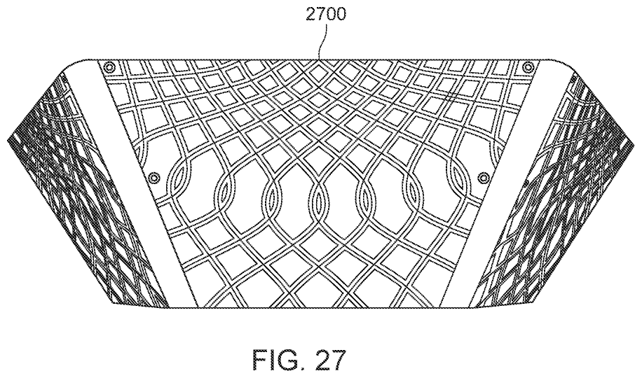

In one version of the enclosure, the enclosure includes replaceable panels (FIG. 27) which can be exchanged with other styles of panels which can vary in aesthetic design and functionality for the sensory stimulation feedback system. There are multiple variations of the replaceable panels including ports for NFC or RFID communication, bidirectional network communication, audio inputs, audio outputs, ambient LED lighting, video displays, etc.

One version of a replaceable panel includes a Near-field communication (i.e., NFC) card reader. This NFC panel allows users to be associated with physical cards which are used with a user management system within the sensory stimulation feedback system. Near-field communication is a set of communication protocols that enables communication between two electronic devices over a short distance. NFC offers a low-speed connection through a simple setup that can be used to bootstrap more capable wireless connections. NFC is based on inductive coupling between two so-called antennae present on NFC-enabled devices.

The sensory stimulation feedback system's processing module includes multiple controllers, connectors, ports, storage connections, as well as electrical traces which route various electrical signals to and from various modules, sources, and destinations. The processing unit is responsible for controlling inputs and outputs, receiving external and internal data, processing data, saving data, storing data internally or externally, executing internal and external programs, controlling the power distribution module, sending and receiving commands to and from attached and/or remote emitters, devices, or systems. In one embodiment, a microphone module, ethernet ports associated with DMX input and output signals, and multiple SD card slots are installed on the processing unit. This processing module is designed to be housed within the enclosure and uses the internal electrical routing distribution hexagon structure to route the different electrical signals to the proper destinations.

In one embodiment, the sensory stimulation feedback system's power distribution module is designed to modify and convert an alternating current power source into the required direct current voltage and amperage specifications required by different sets of emitters through the use of multiple transformers. This module is designed to be housed within the enclosure and uses the internal electrical routing distribution hexagon structure to route the different electrical signals to the proper destinations.

In one embodiment, the sensory stimulation feedback system's internal electrical routing distribution hexagon structure is designed to safely route various electrical signals including emitter control signals, direct current voltage, alternating current voltage, and other data while keeping various types of electrical signals separate. Each of the six different sides of the internal electrical routing distribution hexagon structure routes a specific type of electrical signal to a destination. This structure is designed to be housed within the sensory stimulation feedback system enclosure and provides additional structural stability to the internal modules.

The sensory stimulation feedback system is capable of adapting to various user needs and wants by being designed as a modular system. Modular components can include power distribution modules, processing modules, modular inner units, outer units, cooling units, diffusion units, and others.

The modular inner units are housed in an isolated separate enclosure which allows for safe modular installation and removal. The modularity of the design allows for a user to install or remove modular components to the system. Once a modular component is connected and/or installed, the sensory stimulation feedback system can receive data from, send data to, control, and/or be controlled by the connected hardware through software commands.

The External Modular Interface Controller (EMIC) is designed to interface the system with external devices such as emitters, sensors, and external systems while through a variety of hardwired and wireless communication protocols.

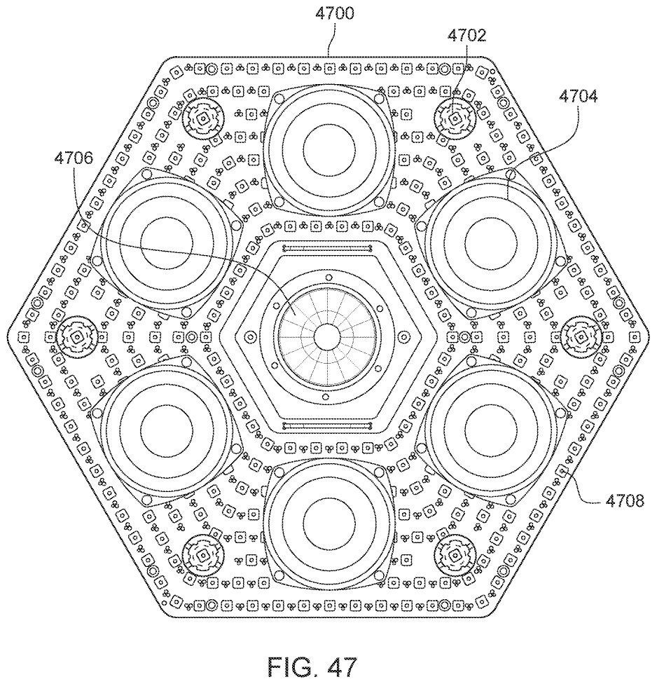

In one of the possible configurations of the sensory stimulation feedback system, the outer unit includes multiple speakers (FIG. 47). When this unit is paired with or placed into an array of other sensory stimulation feedback systems equipped with speakers, it can provide various audio experiences (e.g., binaural beats) for users.

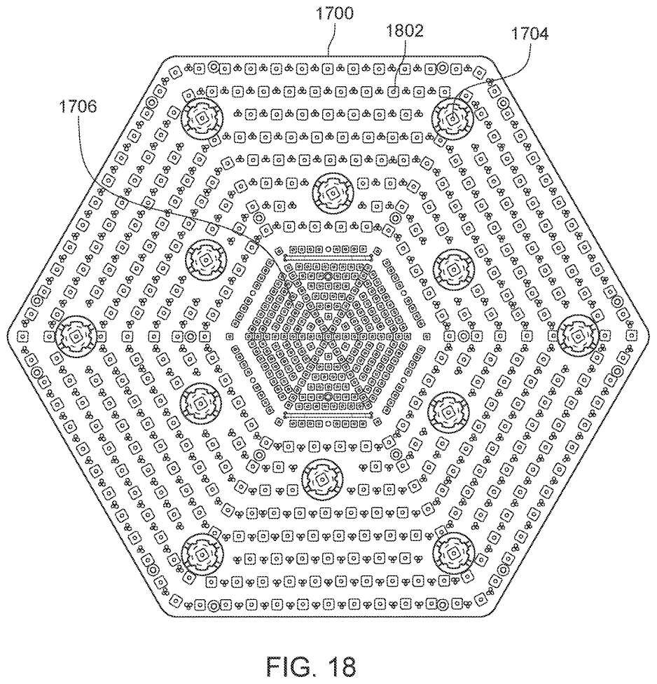

In another configuration, the sensory stimulation feedback system consists of multiple arrays of LEDs (FIG. 18). These arrays are designed to be controlled by the processing module in order to stimulate a user's optic nerve. When the user's optic nerve is stimulated in this way, they can experience an altered state. In addition to the altered state achieved through their sense of sight being stimulated, the processing unit can also control emitters and systems associated with the user's other senses.

In yet another embodiment, the outer unit is designed to house a smaller internal light (source light A) (which contains an array of LEDs) within it. This configuration is equipped with an outer unit containing an array of RGB LEDs and a modular clear overlay containing phototransistors. These phototransistors sense the activity of the smaller internal light (source light A)'s LED array activity. As the smaller light turns on and begins to display light patterns, the sensory stimulation feedback system begins to record, analyze, and store the data from the internal light (source light A). Based on the variables recorded from the internal light (source light A)'s LED array, the sensory stimulation feedback system adjusts the variables (e.g., color, strobe frequency, etc.) for the RGB LED array located on the outer unit of the sensory stimulation feedback system and/or sends control signals to other devices or systems.

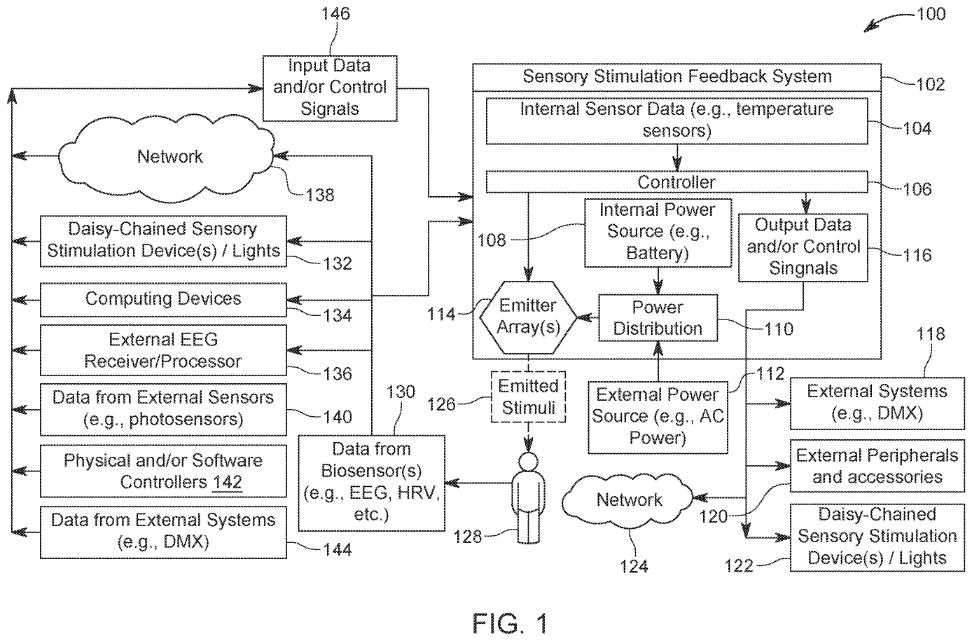

FIG. 1 is a block diagram illustrating functional components of a system according to an embodiment of the subject matter described herein. Referring to FIG. 1, the system 100 may include an emitter array 114, such as an array of light-emitting diodes (LEDs) or other emitters, which can receive a variety of inputs for controlling the operation of the device, including individually controlling each emitter in the array, and send a variety of outputs to multiple sources.

The sensory stimulation feedback system 102 includes an enclosure which houses a controller module 106, an internal power source module and/or an input for an external power source 108, internal sensors 104, connections for modular emitter outputs, connections for a cooling module, and may have a front face for physically securing a plurality of emitters (e.g., LEDs) in a fixed arrangement. As such, when activated, the plurality of emitters is configured to emit electromagnetic radiation (e.g., light) in substantially the same direction. This direction is perpendicular to the plane of the front face such that the light shines in a beam toward a user when the user is located under the front face of the system.

Various components or sub-assemblies may be associated with the front face of the system and configured for supporting the operation and type of the emitter array. These components may include a power source 112 for providing energy sufficient to activate the emitters, a heatsink or other heat dissipation means for cooling the emitters, a controller for controlling the operation of the emitters, a housing that can be detachably coupled to the front face to form the device, a diffusion layer for the emitters, and a cooling module. When assembled, the device may enclose an interior volume that includes one or more components of the device including, but not limited to, wires, anodes, cathodes, batteries, heatsinks, logic circuits, processors, and memory. This allows the device to be easily transportable as well as compatible with physical devices, such as stands, clamps, hooks, etc. for securing the device in a desired location and orientation.

While the system may be pre-programmed to display one or more patterns via an emitter array, the system may also receive one or more inputs for controlling or adjusting the operation of the emitters in the array. In one embodiment, the input may include a waveform, multiple waveforms, or other signal received from an electroencephalogram (EEG) device 130. The EEG device 130 may be associated with a user 128.

The user associated with the EEG device may also be simultaneously exposed to the electromagnetic emissions of the device. For example, the effect of the sensory stimulation feedback system on the user may be monitored and recorded by the EEG device and fed back as input to the system for adjusting its operation, creating a closed loop feedback system.

Alternatively, the user associated with the EEG device may be a different user than the user exposed to the electromagnetic emissions of the sensory stimulation feedback system. For example, a waveform or other signal may be recorded by monitoring a first user via the EEG device. This EEG information from the first user may be used as input to generate a light pattern for display on the sensory stimulation feedback system. The generated light pattern may then be observed by a second user.

The sensory stimulation feedback system may also be controlled with a physical controller. The physical controller can control and change variables (e.g., brightness, strobe frequency, color, etc.) to create new programs and/or create and modify pre-programmed programs, real-time sensor generated sensory experiences (e.g., EEG data driven experiences), and/or hybrid sessions.

Additionally, the system 102 may include a power distribution module 110 for powering the components. The system may also output data and/or control signals 116 to external systems 118, external peripherals and accessories 120, and other daisy-chained sensory stimulation devices 122. Similarly, data from biosensors 130 may be provided as feedback to a variety of devices including others daisy-chained sensory stimulation devices 132, computing devices 134, EEG receiver 136, network 138. This information may be combined with information from photosensors 140, physical and/or software controllers 142, and data from external systems 144 and provided as input data and/or control signals 146. These input data and/or control signals 146 are then provided back to system 102.

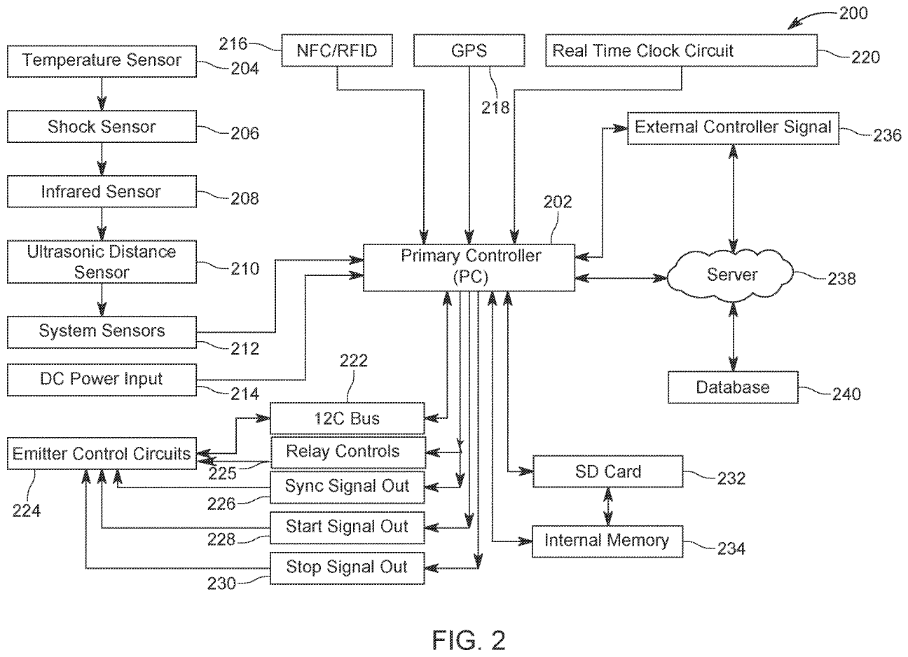

FIG. 2 is a block diagram of the primary controller circuit of a sensory stimulation system, according to an embodiment of the subject matter described herein. diagram illustrating an embodiment of the Primary Controller (PC) 202 according to an example of the subject matter described herein. Referring to FIG. 2, the Primary Controller Circuit 202 is responsible for interfacing with various modules and components connected by a variety of wired and wireless protocols.

The Primary Controller Circuit's function within the system is to provide an entry point into the system, to store and retain data VIA the memory storage options within the circuit or by external means, to automatically obtain, set, and retain the system time using the GPS antenna 218, NTP server 236, or by other means, to read, write, and/or modify data located on RFID and/or NFC devices 216, to receive, store, and monitor sensor data from internal sensor inputs 212 and external sensor inputs 204, 206, 208, 210, to communicate to Emitter Control Circuits (ECC) 224 wirelessly and through hardware busses 222, to enable the ECC array through individual relays 225, to ensure accurate timing and system safety for each ECC through the activation of dedicated start 228, stop 230, and sync connections 226, to receive control signals through wired and wireless transmissions which can be transmitted to the ECC array.

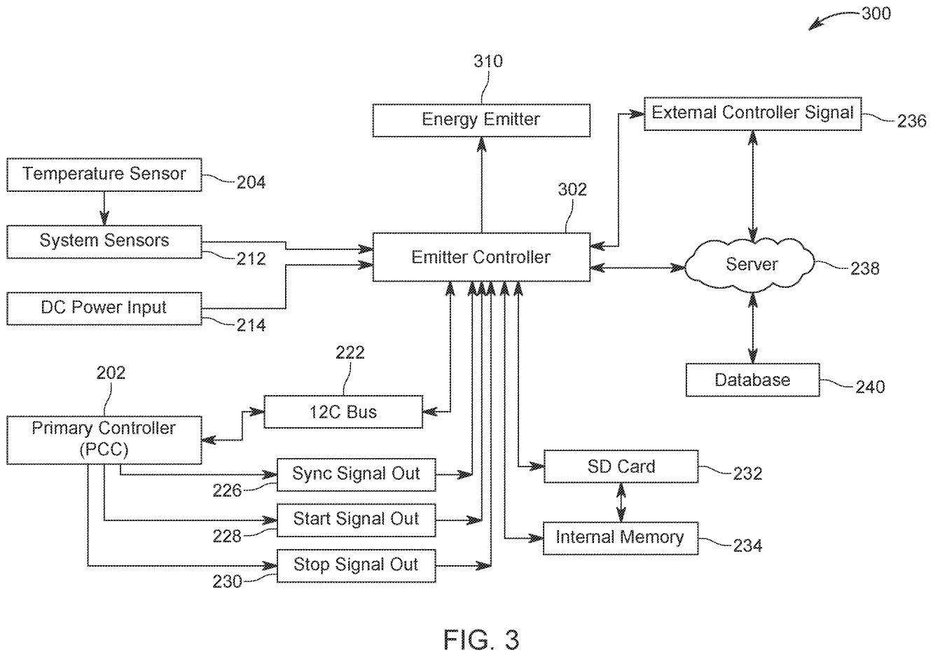

FIG. 3 is a block diagram of an emitter controller circuit of a sensory stimulation system, according to an embodiment of the subject matter described herein. The diagram illustrates an embodiment where Emitter Controller (EC) 302 is responsible for interfacing with the Primary Controller (PC) 202 and other systems to receive control signals which are used to control the variables associated with the connected energy emitter 310.

The Emitter Controller Circuit's function within the system is to receive control signals from the PCC, the server, external controllers, and other sources through wired and wireless transmissions to provide ECC specific and/or system data, including connected or associated energy emitter specific variable control values, to store and retain data VIA the memory storage options within or outside of the circuit through wired or wireless connections, to automatically obtain, set, and retain the accurate system time provided by the PCC 202, to receive, store, and monitor sensor data from internal and external sensor inputs, to control the associated connected energy emitter 310 through hardware busses, to ensure accurate timing and system safety for the user, each ECC can be triggered uniformly through the activation of dedicated start, stop, and sync connections from the PCC 202.

The Emitter Controller Circuit's function within the system is to receive control signals from the PCC 202, the server 238, external controllers 236, and other sources through wired and wireless transmissions to provide ECC specific and/or system data, including connected or associated energy emitter specific variable control values, to store and retain data VIA the individually accessible memory storage options within or outside of the circuit through wired or wireless connections, to automatically obtain, set, and retain the accurate system time provided by the PCC, to receive, store, and monitor sensor data from internal and external sensor inputs, to control the associated connected energy emitter through hardware busses, to ensure accurate timing and system safety for the user, each ECC can be triggered uniformly through the activation of dedicated start, stop, and sync connections from the PCC.

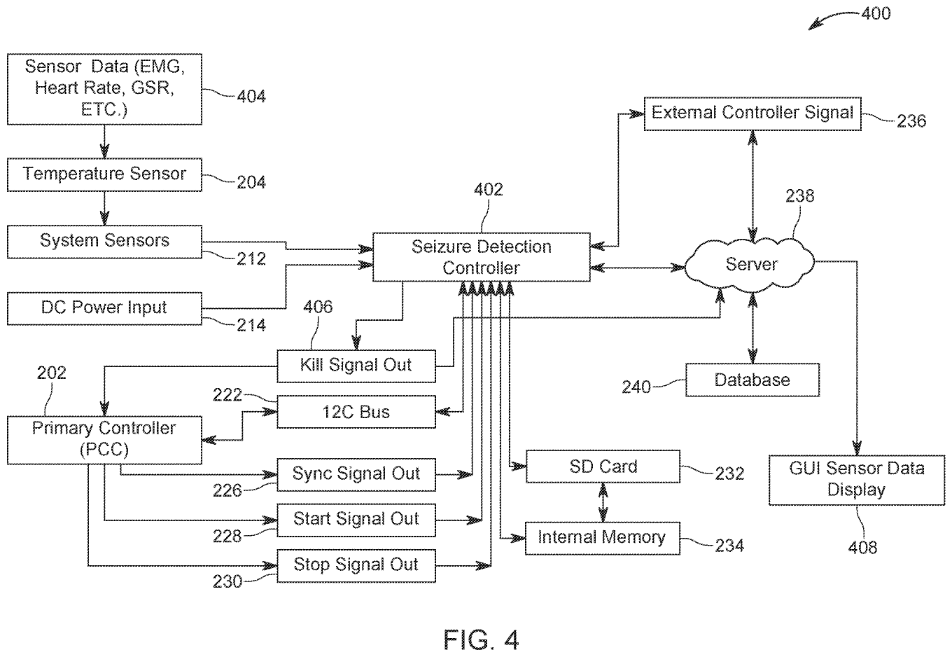

FIG. 4 is a block diagram of a seizure detection controller circuit of a sensory stimulation system, according to an embodiment of the subject matter described herein. The diagram illustrates an embodiment 400 of one type of safety device, the Seizure Detection Controller Circuit, according to an example of the subject matter described herein. Referring to FIG. 4, the Seizure Detection Controller (SDC) 402 is responsible for obtaining, storing, and monitoring the user's or users' biological sensor data 404 as well as environmental sensor data 204. If biological irregularities are detected, the SDC 402 sends a “kill switch” signal 406 to the PCC 202, each individual ECC 236, the server 238, any active controller or controllers, and other connected peripheral devices, causing them all to instantly stop their emissions and flag the most recent variable values. All the sensor data is uploaded and retained, which is compiled into that session data, regardless of whether or not a kill switch signal had been triggered or not.

The Seizure Detection Controller Circuit's 402 function within the system is to provide an additional safeguard for the user's experience as well as peace of mind for the administrator operating the system. When functioning within the context of the closed feedback loop, the SDC 402 is required for the system to operate. In other operational configurations, the SDC 402 can be optional as opposed to required. As the SDC 402 receives the various streams of sensor data from the sensors 404, 204, 212, it internally and/or externally stores, transmits to the database 240 via the server 238, where the data is monitored, displayed, and analyzed to detect potential discomfort and issues the user is experiencing while engaging with the system. If a seizure or other issue is detected, the SDC 402 instantly transmits the kill switch signal 406 through wired and wireless transmissions to the various components of the system (PCC, ECC, server, etc.) which triggers all energy emitters to stop, a remote audible tone through an external sound emitter from the PCC 202. Despite not controlling an energy emitter, the SDC 402 still receives the synchronization signal that the ECCs receive during a session in order to ensure accurate timing. Other peripherals, such as external wired panic buttons can interface with the SDC 402 as additional types of controllers, which will trigger the kill switch signal.

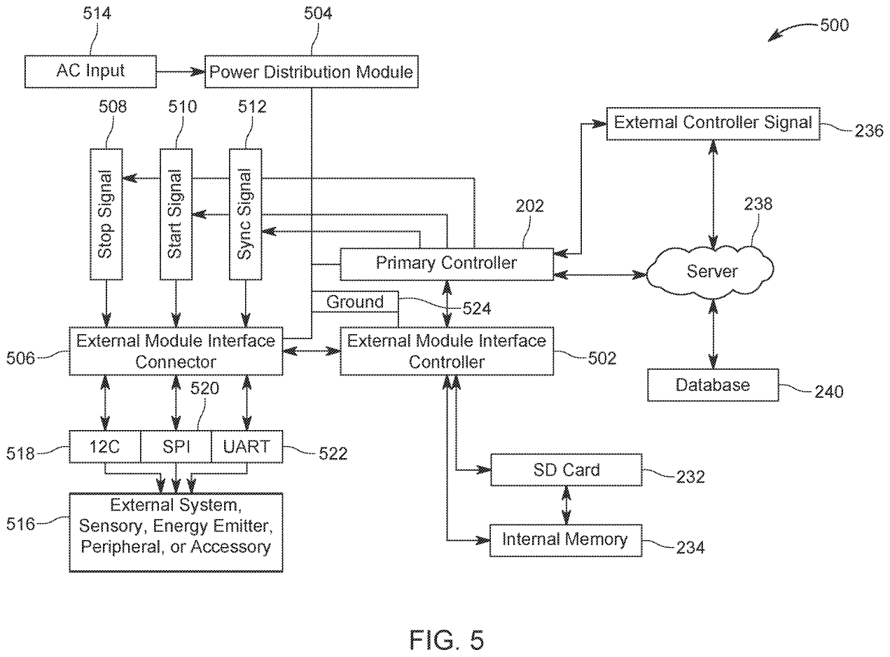

FIG. 5 is a block diagram of an external module interface controller circuit of a sensory stimulation system, according to an embodiment of the subject matter described herein. Referring to FIG. 5, the External Module Interface Controller (EMIC) is responsible for interfacing the hardware connections and handling the data of an external device.

The EMIC is capable of interfacing with various types of sensors, external energy emitters, data storage devices, analog and digital devices, external systems, proprietary accessories, and other peripheral third party devices. The EMIC is capable of utilizing multiple hardwired communication protocols as well as interfacing wirelessly to the external device. The essential aspect of the EMIC is to provide a wired interface between an external device/system, the PCC, and the SDC so that any peripheral, sensor, energy emitter, third party device, or other potential device can be integrated into the system while maintaining safety standards and time calibrations that are required of this technology. The EMIC's ultimate function is to ensure the inclusion of other technologies that may not exist yet or are older, lesser known, more abstract, or reside within a niche market. The EMICs can be “daisy chained” as shown in FIG. 1.6. The EMIC is dynamically calibrated through software specifically depending on the device that is connected to it. The PCC interacts with the EMIC according to how the EMIC is calibrated. For example, the EMIC could be connected to an MRI machine, EEG headset, an array of ultrasonic transducers aligned on the acupuncture meridians, etc.

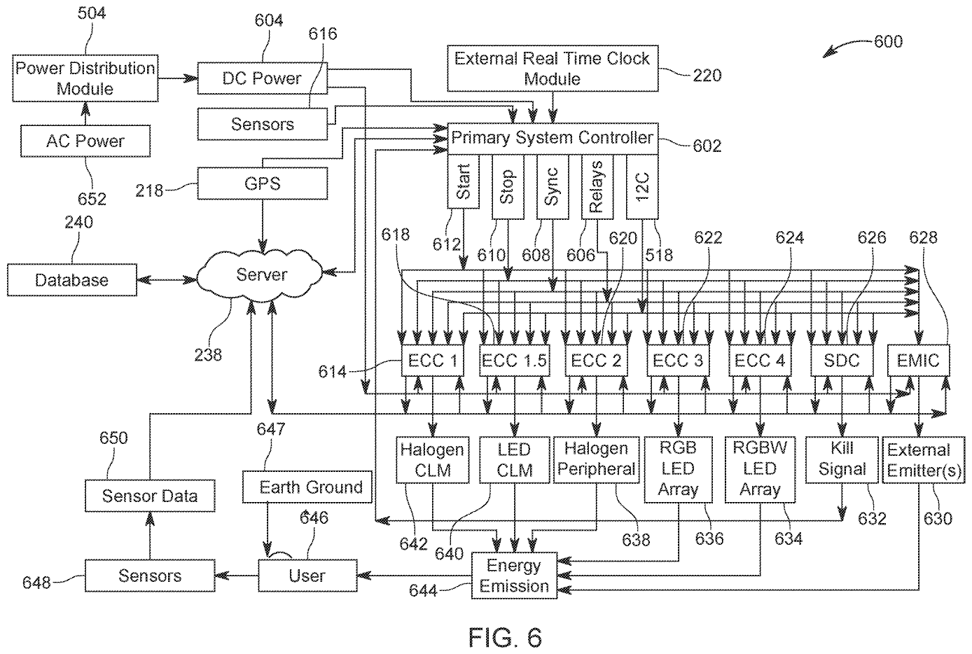

FIG. 6 is a block diagram of a sensory stimulation system, according to an embodiment of the subject matter described herein. a diagram illustrating an embodiment of one type of External Module Interface Controller, according to an example of the subject matter described herein. Referring to FIG. 6, the closed feedback loop system in this embodiment presents a more complete scenario of the closed feedback loop system.

Through the modularity of the Center Modules (CM)s and modular energy emission subsystems, the user experience can be customized. For instance, in FIG. 6, there are two different types of Center Light Modules (CLMs) represented by ECC 1 614 and ECC 1.5 618. These modules are self-contained and are interchangeable with other CMs. Within the ECC 1 to ECC 1.9 range, there can only be a single CM present at a time due to the Pauli exclusion principle. The modularity of just this one module expands the potential for the user experience exponentially.

In FIG. 6, the optical stimulation possibilities from only the light emitting emitters are one value difference of one of the following, the LED CLM, the Halogen Peripherals, the 372 RGB LED array, and the 12 RGBW LED array. This diagram also illustrates the addition of the user being connected to an Earth Ground 647, which is included in an effort to minimize any potential of static electricity from affecting the calibration and the integrity of any data collection from the sensors or discharging and damaging a connected sensor.

Additionally, this diagram shows the system's use of a synchronized time constant from external real time clock module 220 time to have the PCC, all ECCs, the SDC, the server, and all other devices to accurately to keep and acquire time to be synced to the microsecond or better.

In another embodiment of the closed feedback loop system, the system can be calibrated into the full sensory closed feedback loop by configuring the various components assigning specific energy emitters that are designed to artificially stimulate specific senses in a controlled manner based on the user's biological responses to the introduced artificial stimuli. For example, there may be at latest one emitter for each type of sense: sound, sight, touch, smell, taste. ECC 1 is controlling the unfiltered halogen CLM, ECC 2 is controlling the audio energy emitter, ECC 3 is controlling the RGB LED array, ECC 4 is controlling the RGBW LED array, each of the EMICs are configured differently to either emit energy, connect to an external sensory stimulation system, or integrate a type of sensor into the system. In this embodiment, EMIC 1 is calibrated as a tactile energy emitter, EMIC 2 is calibrated as a gustatory emitter, EMIC 3 is calibrated as an olfactory energy emitter, EMIC 4 is calibrated as an additional audio energy emitter, EMIC 5 is calibrated as an additional sensory input and in this specific embodiment as an external EEG system. It is relevant to note that referencing the sense of touch is not limited to only tactile energy emissions. Within this system's context, in addition to pressure, touch includes multiple types of sensations as they relate to the skin and body feeling which would allow an EMIC to be calibrated to control fans to simulate the sensation of air flow across skin or an automated acupuncture style solenoid system to stimulate different areas of the body, or to the temperature control of a sensory deprivation chamber or a float tank by way of interfacing with the thermostat.

By constantly monitoring the user's brain wave activity, heart rate, and other biological reactions that can provide data, the system is capable to modify the values of any and all ways. By analyzing previously obtained data from the specific user and data from other users, it is beneficial—while being constantly monitored by the SDC and the external EEG system—for the user to put them into a theta state using the combined effects of the unfiltered Halogen CLM and the strobing effects from the two different LED arrays controlled by ECC 3 and ECC 4, while using ECC 2 to emit binaural beats through audio energy emitters.

It is also beneficial in a different scenario to calibrate EMIC 6 to control and be controlled by an external virtual reality system where the virtual reality program or scenario triggers different values for the other connected emitters and in a scenario that is calm and comforting or one that allows a user to safely face their fears (real or imaginary). Calibrating EMIC 1 to control a vibrational table that allows for surface temperature control as well as the frequency of the vibrations of the table to foster a calm relaxing state, while ECC 2 is calibrated to a multi axis audio energy emitter and uses the connected energy emitters to generate user specific binaural beats to put the user into an altered state, if EMIC 2 is calibrated to an external gustatory system, it could be used to provide the taste sensations of a comfort food while the olfactory system calibrated to EMIC 3 reinforces that taste with the smells associated with it, EMIC 4 could be calibrated to an audio energy emitter that plays pre-recorded or live audio affirmations through a microphone input into the calibrated EMIC 4. As the experience progresses, the user would be able to be put into any sort of virtual experience and those experiences would be able to be processed by the user's brain based on the awareness that their five senses provide through their actual artificial stimulations. As the sensory stimulations are validating the way their brain is processing the other sensory stimulations, the experience can become more and more realistic to the user as additional EMIC units or systems are added to the initial system.

One benefit this system could have would be in relation to eye movement desensitization and reprocessing (EMDR). The ability to consistently artificially stimulate a user in the exact same ways, and using data to not only validate the user's real time reactions but to use that data to see changes in the session over time, would be beneficial.

Additionally, since the system can be controlled in a variety of ways, multiple systems can be connected together VIA the server's control, other PCCs, external controllers, and other ways, the potential for expansion is never ending.

In another embodiment, the system can be streamlined to stimulate a single sense. For example, ECC 2 may be configured to emit audio energy emissions, resulting in a binaural beat experience for the user. Although the other emitters are present, they are not and do not need to be active for the system to function. Within this embodiment, the closed loop system monitors the user's EEG and other sensory data, sends the data to the server, the machine learning model analyzes the data, in this embodiment, the software on the server automates the audio emitter's variable values (frequency, volume, etc.) based on the sensor data, resulting in a more effective experience for the user. The session data is then stored and backed up and added to the machine learning's referenced dataset so it can be used and accessed later.

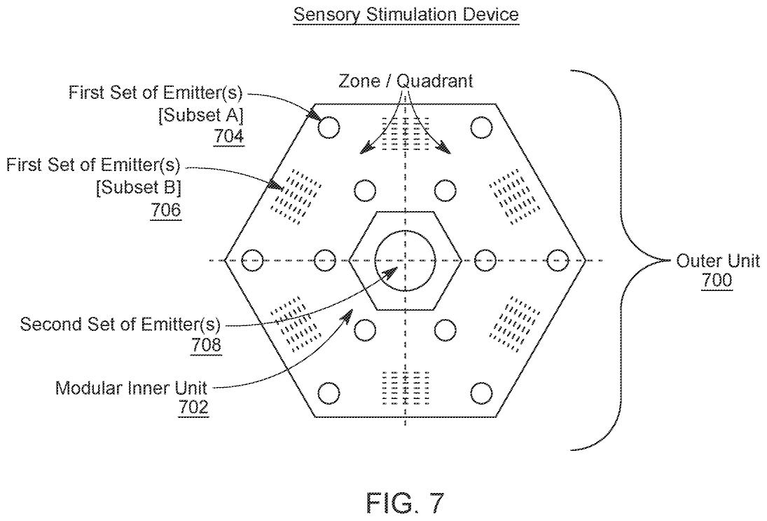

FIG. 7 is a diagram illustrating a sensory stimulation feedback system according to an embodiment of the subject matter described herein. Referring to FIG. 7, the outer front-facing unit 700 is a substantially flat and rigid material having a plurality of perforations for securely inserting a corresponding plurality of emitters therethrough. The set of all emitters associated with the system and secured by the outer unit may hereinafter be referred to simply as “the emitters”. It is appreciated that the emitters may be individually controllable or may be controlled in one or more groups or sets. These logical groupings may be referred to simply as “groups” of emitters.

Additionally, it is appreciated that the emitters may also be physically grouped. For example, three logical groups of emitters may correspond to three different physical types of emitters. This may be useful because different types of emitters may have different characteristics or requirements. In one embodiment, a first set of emitters is arranged in a first plane corresponding to a first set of holes or perforations or locations within the front face of the system. The first set of emitters 704, 706 may include a plurality of multi-color capable light emitters, such as 372 small RGB LEDs. These LEDs may be arranged in a symmetrical pattern within an outer portion of the front face, such as a ring shape surrounding an inner hub portion. The first set of emitters may be associated with a circuit unit for supplying operating power to each emitter in the first set of emitters.

A modular inner unit 702 may be detachably coupled to the outer unit and include a second set of emitters 708 arranged in the same plane as the emitters of the outer unit, when assembled. For example, an inner hub may be removeable and clipped or screwed into a central hole in the outer unit. This allows the modular inner unit to be exchanged for different configurations of emitters in the center of the system. In one embodiment, the modular inner unit may include a plurality of LEDs. The LEDs of the inner hub unit may be the same or different from the LEDs of the outer unit. For example, the modular inner unit LEDs may be RGB (like the outer unit LEDs) or white (different from the outer unit LEDs). The modular inner unit LEDs may be arranged in a different density than that of the outer unit. For example, the modular inner unit LEDs may contain more emitters per unit of surface area (denser) or may contain fewer emitters per unit of surface area (less dense) than the outer unit. In other embodiments, the density of LEDs within the inner and outer units may be the same but the physical arrangement of the LEDs may be different. For example, the outer unit LEDs may be arranged in a grid pattern while the modular inner unit LEDs may be arranged in a concentric circle pattern. The subject matter described herein is not limited to any particular density or other physical arrangement of LEDs of the inner or outer units.

In another embodiment, the modular inner unit may contain a different type of emitter or sensor than the outer unit rather than a different arrangement of the same emitter. For example, the modular inner unit may contain a laser module, an essential oil diffuser, an array of various light sources, a high-powered LED, a camera module, a speaker, or just a single halogen light source that is significantly different from any individual emitter in the outer unit.

Regardless of the type of emitter, the emitters emit energy or matter, whether individually or collectively, electromagnetic radiation (light) having a wavelength between about 1500 nm (infrared) and about 100 nm (ultraviolet), soundwaves (audio, ultrasonic), vapor (essential oil diffuser), etc. While this range of electromagnetic radiation may include wavelengths slightly above or below that of visible light, it is appreciated that such wavelengths (e.g., infrared/near infrared and ultraviolet) and the combination of multiple wavelengths (e.g., the wavelengths from a full spectrum halogen light source) may nevertheless influence the user when experienced by the user. For example, the UV output from an unfiltered halogen lamp in a modular inner unit can penetrate further through the user's skin and into the brain and therefore may intensify the effects of the experience.

The emitters may be associated with a controller for operating the plurality of emitters based on an input. The controller may include one or more logical controllers and one or more physical controllers. For example, the controller may include a hardware logic circuit or a processor and memory executing non-transitory computer-executable instructions.

In some embodiments, the controller may be local to, or integrated with, the sensory stimulation feedback system. This may include enclosing a logic circuit or other electronic hardware within the main body housing. In other embodiments, the controller may be external to, or remote from, the sensory stimulation feedback system. This may include executing the functions of the controller in software on a computing device, such as a computer, server, mobile device, or cloud computing host, which is communicatively coupled to the sensory system. For example, the sensory stimulation feedback system may include a wireless communications interface (WiFi, Bluetooth, cellular) for receiving instructions from a remotely hosted controller or streaming sessions stored on remote servers.

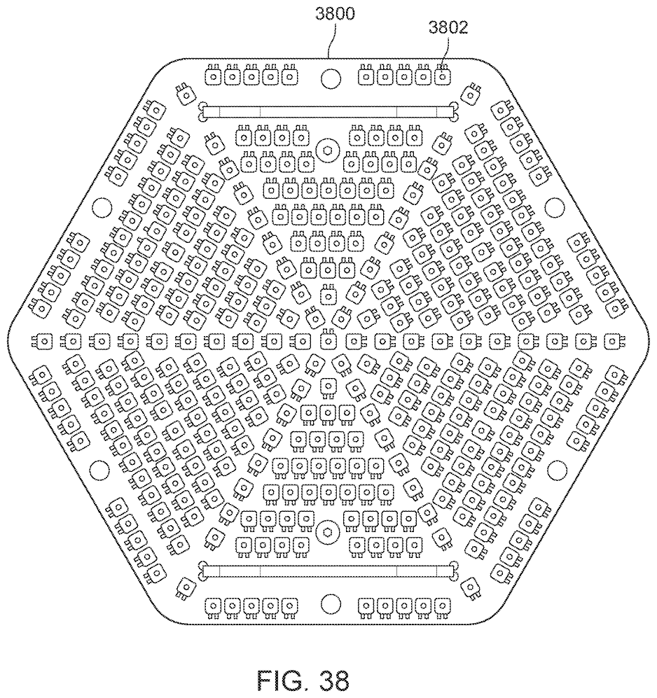

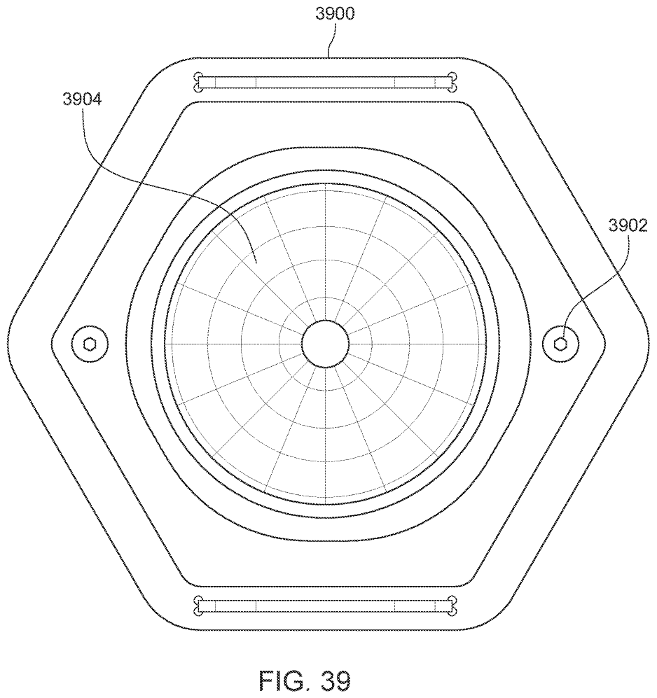

It is also appreciated that in some embodiments, each sensory stimulation feedback system may be associated with its own controller in a one-to-one relationship or, in other embodiments, multiple sensory stimulation feedback systems may be associated with a shared controller (FIG. 38). The latter embodiment may be used in a scenario that includes interconnecting multiple sensory stimulation feedback systems and controlling the operation of the multiple sensory stimulation feedback systems collectively. In such a scenario, the patterns displayed by the group of multiple sensory stimulation feedback systems may be different from the patterns displayed by a single sensory stimulation feedback system. For example (FIG. 39), a shape such as a triangle, may be displayed across seven sensory systems such that one line of the triangle shape is displayed by two sensory stimulation feedback systems, a second line of the same triangle shape is displayed by another two sensory stimulation feedback systems, and so on. As such, the triangle shape of the pattern may only be apparent when the operation of the group of the seven sensory stimulation feedback systems corresponds with their physical arrangement.

In another example, the signal from a controller can simulate animation through the manipulation of control signals to multiple controllers: as the triangle shape is displayed across the seven sensory systems as in the previous example, when that representation of the triangle begins to rotate, the pixels that represent the sides of the triangle systematically turn off and on to simulate movement and extend to the neighboring sensory stimulation feedback system.

This contrasts with displaying the entire rectangle shape by each sensory stimulation feedback system in a repeating pattern (FIG. 38). Each of these exemplary patterns (i.e., a large, non-repeating triangle spread across all seven sensory stimulation feedback systems or seven small repeating rectangles corresponding to each of the seven sensory systems) may include the same rectangle shape and may be displayed by all seven sensory systems, but when viewed across the group of systems may be referred to as different “patterns” because each pattern would activate a different pattern of individual emitters.

Operating the sensory stimulation feedback system includes triggering, activating, or otherwise controlling the operation of one or more emitters in the array or sending control signals out to external emitters and/or systems. This includes determining a value for at least one variable of (for lighting) for example: a color, an intensity, a duration, a strobe effect, a strobe frequency, and other variables such as waveform type (sine, square, triangle, etc.), duty cycle, and other effects like phase shifting, and algorithm variable selection.

In one embodiment, at least one of the emitters in the array includes an LED and, more specifically, an RGB LED. A light emitting diode (LED) is a type of diode that is specifically designed to emit light. An LED emits light because of the semiconductor material used. In contrast to single-material diodes, LEDs may be made from compound semiconductor materials like gallium arsenide, gallium phosphide and other semiconductor materials that emit light.

An LED can be forward biased or reverse biased, yet an LED may only emit light when in a forward bias condition. To forward bias an LED, its cathode terminal is connected to the negative side of an external bias voltage and its anode terminal is connected to the positive side of the external bias voltage. When an LED is forward biased, the free electrons gain enough energy to cross the junction, recombine with holes, and release energy in the form of light.

The following table illustrates various LED materials and the associated wavelengths of light they produce.

| Color | Wavelength Range (nm) |
|---|---|
| Ultraviolet | <400 |
| Violet | 400-450 |
| Blue | 450-500 |
| Green | 500-570 |
| Yellow | 570-590 |
| Orange/Amber | 590-610 |
| Red | 610-760 |
| Infrared | >760 |

In addition to single-color LEDs, multi-color LEDs may also be used. A red/green/blue (RGB) (and red/green/blue/white (RGBW)) LED is an LED, or LED package, that can produce many different colors. RGB LEDs have three internal LEDs (Red, Green, and Blue) that can be combined to produce almost any color output. However, the intensity of each internal LED may be different in order to produce different kinds of colors when the three color outputs are combined. One method for setting the intensity of each LED in an RGB LED includes pulse width modulation (PWM).

Pulse-width modulation (PWM) is a method of reducing the average power delivered by an electrical signal, by effectively chopping it up into discrete parts. The average value of voltage (and current) fed to the load is controlled by turning the switch between supply and load on and off at a fast rate. The longer the switch is on compared to the off periods, the higher the total power supplied to the load.

Because RGB LEDs have three LEDs inside the LED package, these three internal LEDs may share either a common anode or a common cathode. Thus, RGB LEDs can be categorized as either common anode or common cathode type. In a common anode RGB LED, the common anode is connected to the external anode lead. To control each color, a LOW signal or ground is applied to the red, green, and blue leads and the anode lead is connected to a positive terminal of the power source.

In another embodiment, one or more emitters, such as a halogen light source, may be located within a modular inner unit of the system. The modular inner unit may be modular such that it may be exchanged for other types of modules. Each modular inner unit may have the same or similar physical features in order to be secured within the center portion of the system. This may include a series of pins and/or a magnet tension to keep the modular inner unit securely (and safely) in place and/or threads for screwing the modular inner unit into the system where a corresponding inner ring portion of the outer unit may include threads for receiving the modular inner unit. Alternatively, the modular inner unit may be clamped or otherwise physically secured within a center portion of the system to create a complete assembly.

In another embodiment, the modular inner unit may not have any electrical components, emitters, or circuitry. In this embodiment, the function of the modular inner unit is to protect the enclosure from external dust and debris. This embodiment may include a physical dust filter or screen, decorative faceplate, blank faceplate, or a combination thereof.

The modular inner unit may also include one or more sensors instead of, or in addition to, the one or more embodiments described above. The sensors may include, but are not limited to, thermal sensors, motion sensors, ambient light sensors, microphones, camera modules, and electromagnetic field (EMF) meters.

Power and communication connections may be provided such that the modular inner units and outer units are controllable together as a single device when assembled. In one embodiment, a shared controller may be located within the modular inner unit and provide functionality described herein for both the inner and outer units (e.g., plurality of emitters and/or sensors in the array). In another embodiment, a shared controller may be located within the outer unit and provide functionality described herein for both the inner and outer units (e.g., plurality of emitters and/or sensors in the array). In another embodiment, a shared controller may be located remotely and provide functionality described herein for both the modular inner units and outer units (e.g., plurality of emitters and/or sensors in the array).

In other words, the subject matter described herein includes a central processing unit (CPU) and/or logic circuit that facilitates the localized and/or remote processing and/or routing of data and/or energy within, to, and/or from the core module (hereafter also referred to as “Hub”). The Hub serves as a central physical housing component of the invention, acting as a connection point by which energy emission extensions, containing one or a multitude of energy emitters and/or transducers (hereafter referred to as “Extension” or “Extensions”) can be connected and interconnected interchangeably. In this manner, a range of fixed and/or actuated energy emitters and/or transducers can be combined and added to the primary energy emitter/array.

It is imagined within the scope of the invention that the Hub and/or Extensions may include one or a multitude of energy emitters and/or input/output transducers including, but not limited to halogen lamps, light-emitting diodes (LEDs), electroluminescent coatings, lasers, speakers, transducers, solenoids, valves, motors, actuators, solar cells, thermal sensors, motion sensors, and electromagnetic field (EMF) meters. The Hub and/or Extensions may also include passive and/or active sensory components such as, but not limited to heating and/or cooling systems and/or components which aid in the facilitation of such functionality such as, but not limited to tubing, piping, hoses, and/or other airflow or curvature designs which might aid in desirable sensory effects.

Similarly, the Array may incorporate internal and/or external interactive sensory devices such as, but not limited to tactile transducers, heating and/or cooling pads, actuated platforms, chemical dispersant mechanisms, and/or other environmental control systems that control, are controlled by, and/or work freely in proximity of the Array. Finally, the Array as a whole, the Hub, and/or Extensions may be controlled by a random and/or preset automated template, by an internal and/or external template derived from analysis of other media, by responsive feedback derived from analysis of the individual experiencing the effects, or by a local or remote operator or by the individual experiencing the effects themselves through the use of an integrated hardware or software controller.

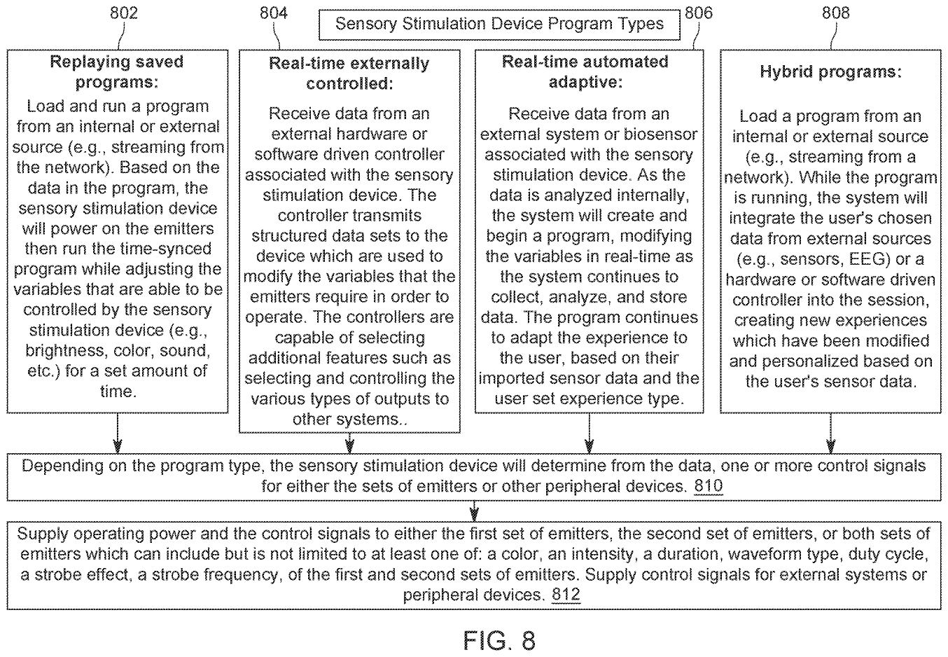

FIG. 8 is a flow chart illustrating exemplary steps of a method for controlling the operation of a sensory stimulation feedback system according to an embodiment of the subject matter described herein. The flow chart may begin by receiving various sensory stimulation program types including replaying saved programs type 802, real-time externally controlled type 804, real-time automated adaptive type 806, and hybrid programs type 808. These are used at step 810 to determine one or more controls signals depending on the program type. At step 812, power and control signals are supplied to the system for execution.

Some of the methods for operating a sensory stimulation feedback system include: loading and running previously created programs from an internal or external source, receiving control data from hardware or software sources which can control the variables associated with the hardware configurations, receiving external or internal sensor data which can be mapped to modify the variables associated with the hardware configurations, and hybrid programs which can modify variables associated with the hardware configurations. Examples of sensor data include: a microphone reacting to an audio signal, EEG input, and biosensor data. Examples of control data include: a pre-programmed routine from a computing device, receiving data from a hardware or software-controlled device, data from another sensory stimulation device, and DMX data; all of which may be received through physical connections to the unit, wired connections (e.g., ethernet), or wireless transmissions such as Bluetooth, WiFi, or other wireless forms of transmission. Next, the controller determines one or more control signals for the first and second sets of emitters based on the one or more inputs. For example, amplitude information of a received input signal may be converted to one or more DMX instructions corresponding to a desired color output, strobe or movement effect, or transitional effect. Finally, the controller supplies operating power and the control signals to the first set of emitters and the second set of emitters in order to control at least one variable including but not limited to: a color, an intensity, a duration, a strobe effect, waveform type, duty cycle, and a strobe frequency of the first and second sets of emitters. For example, a loud portion of an audio signal received as input may correspond to the color red while a quiet portion of the audio signal may correspond to the color yellow, with a continuous gradient therebetween. Additionally, an “excited” EEG input may correspond to high-intensity light output while a “resting” EEG input may correspond to low-intensity light output. The controller may then generate one or more patterns to apply to the emitter array and/or to additional internal or external systems or other devices.

For example, the applied pattern may blend the first and second input signals to generate a combined control signal that is transmitted to all emitters in the array. This may include producing high-intensity red light when both the audio signal is loud and the EEG input is excited while producing low-intensity yellow light when both the audio signal is quiet and the EEG input is at rest, with a continuous gradient therebetween.

In another example, separate patterns may be applied to different groups of emitters. This may include displaying a first pattern to a left side of the sensory stimulation feedback system based on the audio input and displaying a second pattern to a right side of the sensory stimulation feedback system based on the EEG input.

In another example, a user is placed under the sensory stimulation feedback system while connected to an EEG. The operator controlling the experience records a period of the user's brainwave activity without stimulation in order to obtain a base level. After the base level has been recorded and stored, the sensory stimulation feedback system performs a full sweep of the different variables associated with the emitter (e.g., for light: strobe frequency, color, etc.). As the user experiences the various colors and patterns generated internally as a result from the emitters, they flag their favorites by pressing a button on a physical controller. When the button is pressed, it records the variables associated with their perception of the experience in a period of time and their EEG data set. The system then stores the EEG data and the values of the variables into a table to incorporate into the user's upcoming sensory experience. The sensory experience uses the saved values and variations of the values (e.g., harmonics) to generate a personalized experience for the user. The data in the table and EEG data are uploaded to a remote server and are stored to be used as a dataset for Machine-learning and to assist in the generation of future sessions.

In another example, a user is placed under the sensory stimulation feedback system while connected to an EEG with the intention of experiencing a “peak” Theta state. The operator controlling the experience records a period of the user's brainwave activity without stimulation in order to obtain a base level. After the base level has been recorded and stored, the system performs a full sweep of the different variables associated with the emitter (e.g., for light: strobe frequency, color, etc.). As the user experiences the various colors and patterns generated from the emitters, the system flags variable changes that affect the amplitude of the user's Theta brainwaves. The system then stores the EEG data and the values of the variables into a table to incorporate into the user's upcoming sensory experience. The sensory experience uses the saved values and variations of the values (e.g., harmonics) to generate a personalized experience for the user with the intention of putting the user in a state of “peak” Theta. The system begins the sensory experience and as the amplitude of the user's Theta brainwave fluctuates, the system adjusts the variables in order to keep the user in the “peak” state for the requested experience duration. The data in the table and EEG data are uploaded to a remote server and are stored to be used as a dataset for Machine-learning and to assist in the generation of future sessions.

In yet another example, a user wants to be put into a peak Theta brainwave state. The user is attached to an EEG sensor and placed under the sensory stimulation feedback system with their eyes closed. The operator controlling the experience records a period of the user's brainwave activity without the system on in order to obtain abase level. After the base level has been recorded and stored, the first set of emitters [subset A] are set to white and begin slowly strobing at a set frequency. During an additional period of time, this LED array begins to increase the strobing frequency and completes a full sweep of the various frequencies. Once the frequency sweep is complete, the sensory stimulation feedback system records the frequency ranges that affected the user's various brainwaves and programs them into a user specific table. The sensory stimulation feedback system then references the table and integrates the Theta data into a personalized program. The sensory stimulation feedback system then runs the program, using the user reactive Theta frequencies. As this program runs, the system begins to slowly cycle the modular Halogen inner unit in brightness from dim to off and back over time. As the modular Halogen inner unit's brightness is cycled, the user's EEG data is being recorded and as their Theta level rises, the system records the Halogen's brightness level. The sensory stimulation feedback system continues to run the program, now combining the user reactive Theta frequencies and Halogen brightness levels. As this program runs, the system begins to slowly fade in and out the LEDs in LED [subset B] uniformly in a slow cycling of the color possibilities (providing a wash effect). The system continues to monitor the EEG data and adds any change to the user's Theta levels to a table. The system then integrates the LED [subset B] data into the program and based on the user's EEG data, the system manipulates the variables (strobe frequency, brightness, color, etc.) of the program to keep the user in a “peak” Theta state based on their EEG data. As this program runs, the system is able to integrate additional sensory stimulations (e.g., audio) using other systems, emitters, and devices using the same procedure relying on the user's EEG data. For example, if the user's EEG data begins to show a lower Theta state, the system can slowly increase or decrease the variables of the additional sensory stimulation emitter and then compare the frequency with prior data to determine if the frequency change affected the user's Theta state.

It is appreciated that the patterns and combinations of patterns may change over time. For example, a pattern of solid lights for a first time period (e.g., 10 seconds or while a predetermined condition is satisfied) may change to a flashing or stroboscopic light pattern for a second time period.

It is appreciated that in some embodiments, the operation and/or adjustment of emitters in the array of the sensory stimulation feedback system may be performed using machine learning, whether in whole or in part. For example, data collected from EEG devices for multiple users may be analyzed to generate a pattern for controlling the emitters. This may include the color, strobing periodicity, intensity, etc. of each emitter. By using data from multiple users, a general profile may be obtained that is more effective for new users without requiring pre-training or EEG analysis of such users.

In another example, historical data collected from the same user over a time period/multiple sessions may be used to optimize the experience for that same user. While this optimization may be algorithmically pre-programmed or adjusted in real-time by a user, the optimization may also be blended with output from a Machine-learning algorithm.

Machine-learning (ML) is the use of computer algorithms that can improve automatically through experience and by the use of data. Machine-learning algorithms build a model based on sample data, known as training data, to make predictions or decisions without being explicitly programmed to do so. Machine-learning algorithms are used where it is unfeasible to develop conventional algorithms to perform the needed tasks.

In certain embodiments, instead of or in addition to performing the functions described herein manually, the system may perform some or all of the functions using Machine-learning (i.e., “artificial intelligence”). Thus, in certain embodiments, machine learning-enabled software relies on unsupervised and/or supervised learning processes to perform the functions described herein in place of a human user.

Machine-learning may include identifying one or more data sources and extracting data from the identified data sources. Instead of or in addition to transforming the data into a rigid, structured format, in which certain metadata or other information associated with the data and/or the data sources may be lost, incorrect transformations may be made, or the like, Machine-learning based software may load the data in an unstructured format and automatically determine relationships between the data. Machine-learning based software may identify relationships between data in an unstructured format, assemble the data into a structured format, evaluate the correctness of the identified relationships and assembled data, and/or provide Machine-learning functions to a user based on the extracted and loaded data, and/or evaluate the predictive performance of the Machine-learning functions (e.g., “learn” from the data).

In certain embodiments, Machine-learning based software assembles data into an organized format using one or more unsupervised learning techniques. Unsupervised learning techniques can identify relationships between data elements in an unstructured format.

In certain embodiments, Machine-learning based software can use the organized data derived from the unsupervised learning techniques in supervised learning methods to respond to analysis requests and to provide Machine-learning results, such as a classification, a confidence metric, an inferred function, a regression function, an answer, a prediction, a recognized pattern, a rule, a recommendation, or other results. Supervised machine learning, as used herein, comprises one or more modules, computer executable program code, logic hardware, and/or other entities configured to learn from or train on input data, and to apply the learning or training to provide results or analysis for subsequent data.

Machine-learning based software may include a model generator, a training data module, a model processor, a model memory, and a communication device. Machine-learning based software may be configured to create prediction models based on the training data. In some embodiments, Machine-learning based software may generate decision trees. For example, Machine-learning based software may generate nodes, splits, and branches in a decision tree. Machine-learning based software may also calculate coefficients and hyper parameters of a decision tree based on the training data set. In other embodiments, Machine-learning based software may use Bayesian algorithms or clustering algorithms to generate predicting models. In yet other embodiments, Machine-learning based software may use association rule mining, artificial neural networks, and/or deep learning algorithms to develop models. In some embodiments, to improve the efficiency of the model generation, Machine-learning based software may utilize hardware optimized for Machine-learning functions, such as an FPGA.

As mentioned above, brainwave entrainment, also referred to as “entrainment”, “brainwave synchronization”, or “neural entrainment”, refers to the natural synchronization of brainwaves (large-scale electrical oscillations in the brain) in response to the rhythm of periodic external stimuli, such as flickering lights. Different states of consciousness may be associated with different dominant brainwave frequencies. As such, brainwave entrainment may be used to induce a desired state.

One application of the sensory stimulation feedback system disclosed herein includes the potential increase of perceived wellness of people with post traumatic stress disorder (PTSD) or other psychological conditions. For example, a user suffering from PTSD may experience a negative pattern of brain activity when recalling a past trauma. The user may be connected to an EEG sensor to record various aspects of their brain activity while in a positive mood or non-traumatized state. This EEG data may be processed by the controller of the sensory stimulation feedback system to generate a light pattern and/or sensory stimulation that, when experienced by the same user, results in a similar positive mood or non-traumatized state. Then, the user may be asked to recall the past trauma while simultaneously being exposed to the positive light pattern and/or sensory stimulation. By actively counteracting the user's previous brain activity response to recalling the trauma by imposing a new, positive, and personalized brain activity response, the user's future brain activity response to recalling the trauma may be changed (e.g., more positive).

In other, non-therapeutic embodiments, the sensory stimulation feedback system may be used for producing a theater or music light show associated with live or recorded music.

For example, a member of a band playing a live concert may be connected to an EEG sensor that records their brain activity while playing music. This information may be sent to a controller and processed to determine a pattern of light to display to an audience through a sensory stimulation feedback system or an array of systems. The light pattern and color data can then be sent to other types of light through DMX and/or other types of signals. In this way, when the musician is excited about playing a particular song or series of notes, the displayed lights may adjust accordingly and in real time.

Another example stems from the previous one. A band with four musicians are playing a concert where all four have EEG headsets on and the EEG headsets are connected to independent sensory stimulation feedback systems. There are four separate display monitors located at the back of the stage, one for each member. As each member plays, the system analyzes their EEG data and incorporates it into for example, a Mandelbrot Set equation resulting in a constantly changing visual fractal on their respective monitors at the back of the stage. A fifth monitor is located above the center of the stage which is connected to a fifth sensory stimulation feedback system. This fifth system receives an output signal from the other four sensory stimulation feedback systems as well as optionally the mixed audio output from the sound board and generates a combined visual representation of the experience of all four band members and outputs it to the fifth monitor. The data from all five sensory stimulation feedback systems is stored and uploaded to a remote server. Once the data is uploaded to the remote server, it is processed and converted into a sensory experience that the band can offer to their fans as a downloadable or streaming add-on when downloading or streaming their concert audio.

In a similar example, the process of recording EEG inputs and processing the inputs to determine a light pattern to be displayed may be performed ahead of time and stored for future use. As such, the light pattern generated based on a particular musician's brain activity may be replayed for audiences without requiring their presence in real-time.

Another example involves a musician in an unstimulating environment connected to an EEG which is connected to the sensory stimulation feedback system above them. After establishing their EEG baseline levels, they begin to listen to some of their previously recorded songs. As their songs are played, their brainwave activity is recorded and is used to generate and record a multi-sensory experience in close to real time which is emitted from the sensory stimulation feedback system. Once complete, the system stores the data associated with experience and the musician can modify the experience which includes modifying the variables of the session (e.g., color, strobe frequency, etc.) or adding a variety of different sensory stimulations. Once the process is complete, the musician now has a new form of intimate experiential media which they are able to offer to their fans that automatically pairs with the music they listened to when the experience was generated.

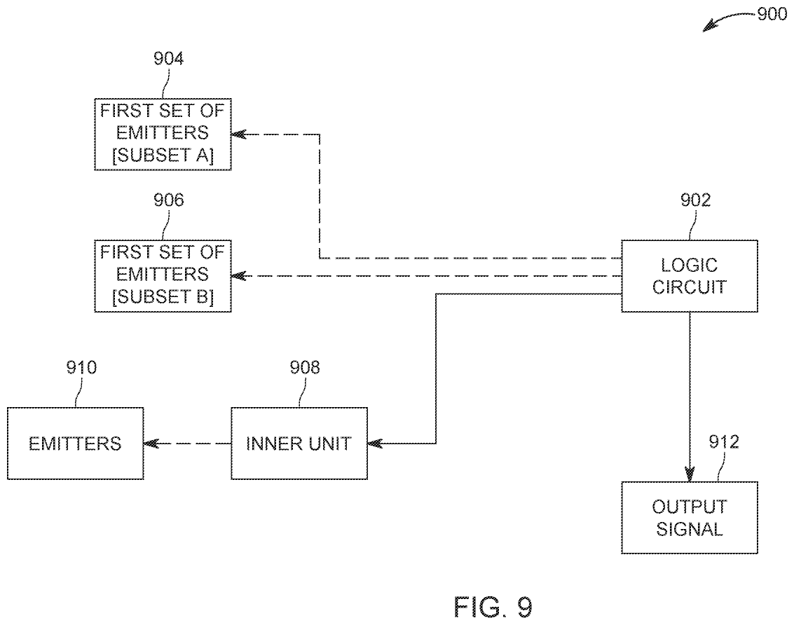

FIGS. 9-14 illustrate various configurations of the sensory stimulation feedback system according to various embodiments disclosed herein. In contrast to conventional configurations which include single-source, single-axis, single spectrum and single-source, single-axis, full-spectrum sensory stimulation (e.g., light emission), the present subject matter includes single-source, full-spectrum and multi-axis emission. Additionally, the present subject matter includes controlling one or more sets of emitters based on one or more received input signals and/or control signals.

FIG. 9 is a diagram illustrating an exemplary configuration of a sensory stimulation feedback system for controlling two sets of emitters using an internal pre-programmed file without any external input signals or external control signals according to an embodiment of the subject matter described herein. Referring to FIG. 9, an exemplary configuration of the logic flow is illustrated in which the logic circuit (902) uses the internal emitter control signals to control the first set of emitters [subset A] (904) and the first set of emitters [subset B] (906). The logic circuit simultaneously sends program control signals to the modular inner unit (908) and if an output is selected, the logic circuit sends an output signal (910) to a specified destination. The modular inner unit then processes the program control signal and controls the emitters (912) on the modular inner unit.

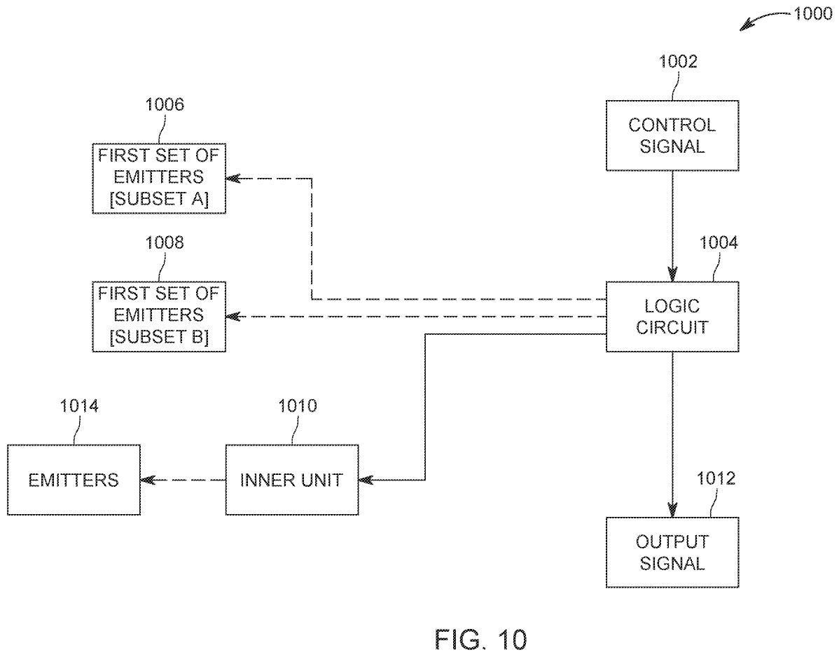

FIG. 10 is a diagram illustrating an exemplary configuration of a sensory stimulation feedback system for controlling two sets of emitters using one received control signal (e.g., a signal from an external software paired sensory stimulation feedback system) and no input sensor signals according to an embodiment of the subject matter described herein. Referring to FIG. 10, an exemplary configuration of the input and output flow is illustrated in which a singular input control signal (1002) is received by the logic circuit (1004), passed on to the modular inner unit (1006) and the output signal (1008), and is processed into an internal emitter control signal which is used by the logic circuit to control the first set of emitters [subset A] (1010) and the first set of emitters [subset B] (1012). The modular inner unit processes the control signal and uses it to control the emitters (1014).

In the case of FIG. 10, a sensory stimulation feedback system 1000 and a modular inner unit's ability to receive a control signal may depend on if the hardware detection circuit has been enabled on each of the systems and has been paired with other hardware.

Control Signals 1002 are inbound data that has been formatted specifically in the language of the code running the sensory stimulation feedback system disclosed herein and the control signals can be received, interpreted, and acted upon by the system. Control Signals may alter the state or behavior of one or many aspects of the system (such as global actions, a specific function of a specific piece of hardware attached to the system, a UI, ON/OF states, etc.). Similar to MIDI, DMX, etc., this signal would conform to specific formatting.

While the Control Signal will tell the slave system what the master system wants it to do, pass on a master control signal to another slave system, or pass data to a master system, the input control signal is just raw signal that can be detected and monitored for use by the machine in some way determined by the operator.

One example of the receiving and using both input signals and control signals includes receiving a control signal from an internet-based master controller instructing the system to look to a specific input signal port on the receiving system, seek a sine wave pattern, then synchronize the peaks and troughs in that signal to the receiving system's output pulse patterns, thus producing a sine wave that is similar to the input signal's pattern.

Whereas the control signal may have a specific format, the input signal is any signal that is detected by the input signal port sensor (e.g., flipping a light switch on and off may be detected as a binary pattern of zeroes and ones by a light sensor as an input signal). As will be discussed below, the input signal(s) and the control signal(s) can work independently, or together, in various ways.

Input signals can include, but are not limited to: sensor data, controller data, external data, audio data, WiFi data, and Bluetooth data.

The Sensor Data may include data produced by, or associated with, one or more sensors. Exemplary sensors include a heart rate sensor, a temperature sensor, and an EEG sensor. In one example, once the sensor data is generated and defined as input data to the sensory stimulation feedback system, the sensor data may be transmitted to a controller (or one of the sub-controllers/processors) associated with/within the sensory stimulation feedback system. The data may then be logged (recorded) and, depending on the type of sensor, interpreted by the system. Next, the sensor data may either be used for adjusting the variables of a sensory experience or light session (pattern applied to the emitter array) or the sensor data may be used to ensure the system is functioning safely.

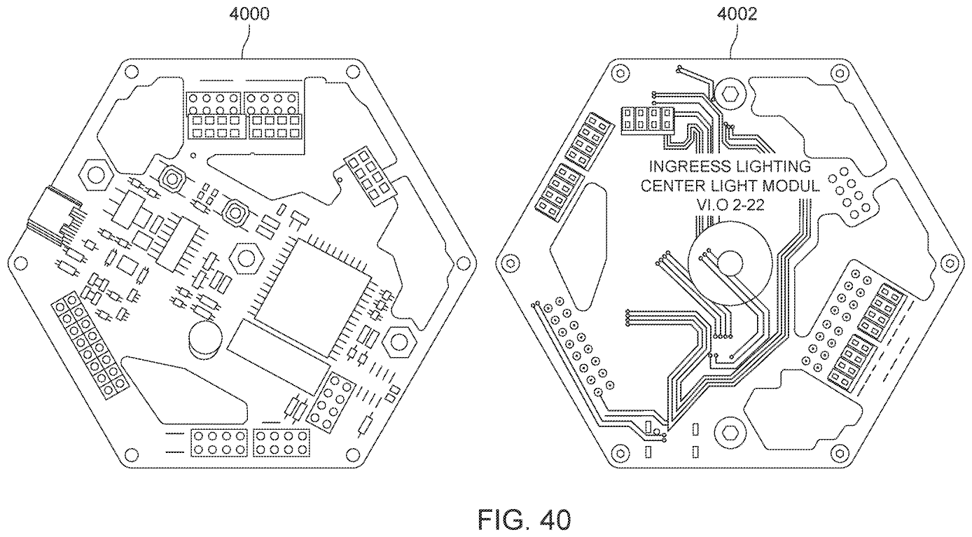

The Controller Data may include data produced by, or associated with, one or more human-computer interface devices (controllers operated by a user). It is appreciated that various types of tactile controllers may be used without departing from the scope of the subject matter described herein. This includes a “Multi-Theremin Controller” (FIG. 40). These controllers may communicate with the sensory stimulation feedback system via one or more communications protocols and associated connections, whether wired or wireless (e.g., Bluetooth). Once a controller is connected (communicatively coupled) to the sensory stimulation feedback system, the controller may send data to the sensory stimulation feedback system which the system will interpret and use for adjusting the operation of the system.

The External Data may include data produced by, or associated with, one or more existing data protocols, such as DMX. In one embodiment, when external data is the signal received as an input, the sensory stimulation feedback system may act as either a “master” system or as a “slave” system.

The Audio Data may include data produced by, or associated with, one or more audio devices or sources. Such audio data can be received by the sensory stimulation feedback system via one or more ports or other input means. For example, inputs to the sensory stimulation feedback system for receiving audio data can include a line level 3.5 mm jack, a microphone, or a Bluetooth receiver. The sensory stimulation feedback system receives the audio signal and interprets the audio waveform. Based on the waveform, the sensory stimulation feedback system can interpret the audio signal (e.g., bandwidth, decibels, etc.) and adjust the operation of the sensory stimulation feedback system accordingly. The sensory stimulation feedback system can also then send the output signal to the 3.5 mm audio output jack, to a connected audio module, and/or via Bluetooth.

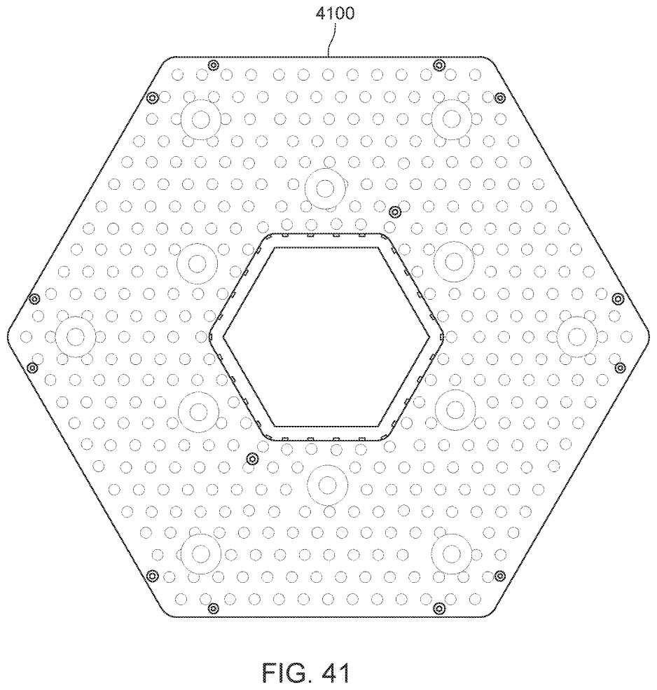

The WiFi Data may include data produced by, or associated with, one or more WiFi-enabled wireless devices. In one embodiment, the primary protocol used for communicating with the sensory stimulation feedback system is through WiFi, including over the Internet. The subject matter herein may include graphical software online (GUI (i.e. Graphical User Interface) presented via a web interface (FIG. 41)) which allows users to create, modify, and save light patterns and send them to the sensory stimulation feedback system. “Master” data received by the system in this manner may be both an input and output signal. In some embodiments, multiple sensory stimulation feedback systems can be “chained” together through a “Master”/Slave” configuration physically or via WiFi. But it is appreciated that other protocols may also be used. Additionally, a user can stream sensory experiences that are being created remotely live or download previously created sensory experience files from an online marketplace.

The Bluetooth Data may include data produced by, or associated with, one or more Bluetooth-enabled wireless devices. In one example, the sensory stimulation feedback system may receive initial programming and OTA (over the air) updates via Bluetooth in addition to receiving and sending an audio signal via Bluetooth.

Output Signals 1012 include signals transmitted by the sensory stimulation feedback system to an external or remote device. Output signals include, but are not limited to: DMX signals, WiFi signals, Bluetooth signals, and Audio signals.

DMX output signals are signals associated with the DMX protocol. When the sensory stimulation feedback system is set as the “Master” in a DMX-based context, the sensory stimulation feedback system may transmit DMX output data to the other sensory stimulation feedback systems or other DMX enabled devices in the same DMX-based context, thereby controlling them.

WiFi output signals are signals associated with the WiFi protocol. As mentioned, the sensory stimulation feedback system may be controlled via WiFi locally or through the internet. The sensory stimulation feedback system issues a “handshake” back to online hosted software which lets the software know that the system has received the data it was sent. In embodiments where the sensory stimulation feedback system is designated as the “Master” and there are other sensory stimulation feedback systems present, the “Master” sensory stimulation feedback system can send out the same (or individualized) signals to the “Slave” sensory stimulation feedback systems, thereby controlling them. A user can create and choose to upload or stream a light session through the internet to others around the world through an online marketplace.

Bluetooth output signals are signals associated with the Bluetooth protocol. The system can transmit audio and other data VIA Bluetooth. The sensory stimulation feedback system may provide “handshakes” to the sensory stimulation feedback systems or other devices it is connected to ensure the devices are paired and that data has been received.

Audio output signals are signals associated with any audio protocol. The sensory stimulation feedback system can send an audio output to an external device. For example, via additional physically connected hardware, the 3.5 mm jack, or via Bluetooth as previously mentioned.

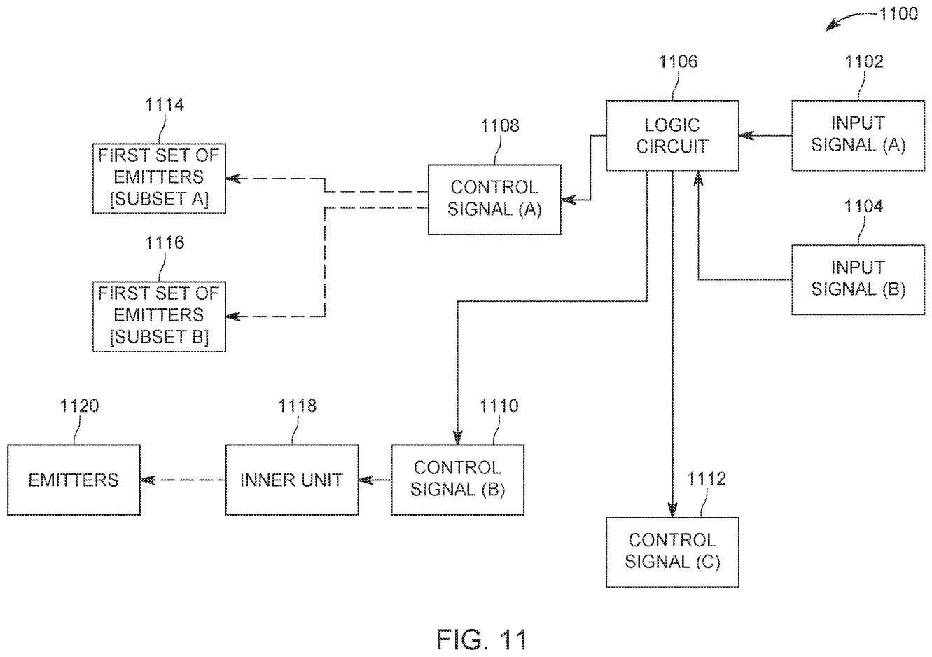

FIG. 11 is a diagram illustrating an exemplary configuration of a sensory stimulation feedback system for controlling two sets of emitters using two received input signals that are converted into control signals according to an embodiment of the subject matter described herein. Referring to FIG. 11, an exemplary configuration of the input and output flow is illustrated in which two input signals marked as input signal A (1102) and input signal B (1104) are received by a logic circuit (1106) and where the logic circuit receives and processes the two input signals and transmits a new set of control signals marked as control signal A (1108), control signal B (1110), and control signal C (1112). Control signal (A) (1114) is the data required for controlling the first set of emitters [subset A] (1116) and the first set of emitters [subset B] (1118). Control signal (B) (1120) is the data required for controlling the modular inner unit (1122). Control signal (C) (1124) is the now processed data that can be sent to other devices such as an output signal to additional sensory stimulation feedback systems. The modular inner unit (1126) then processes control signal (B) and uses the data to control the attached emitters (1128).

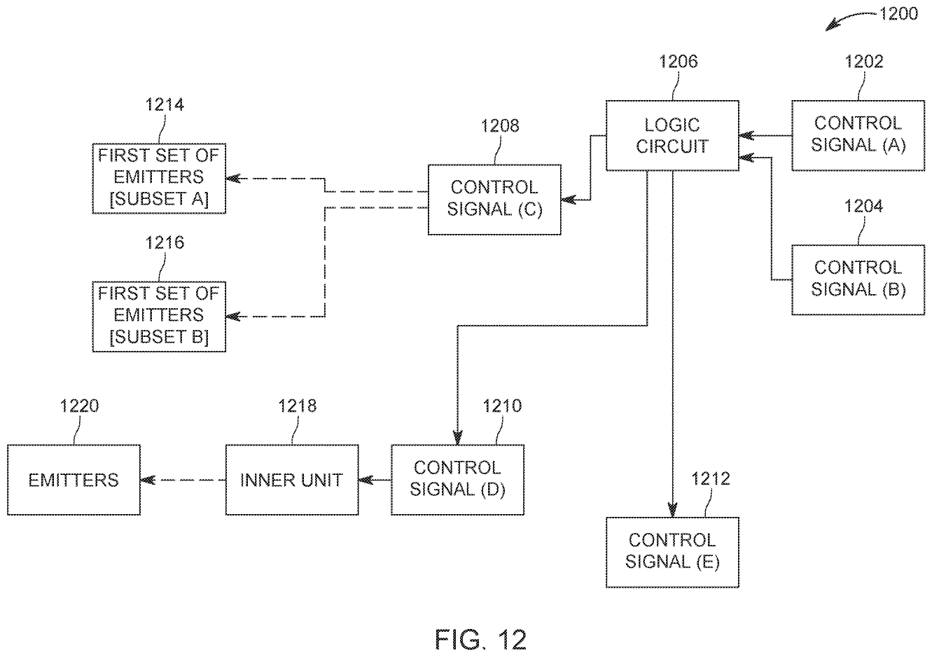

FIG. 12 is a diagram illustrating an exemplary configuration of a sensory stimulation feedback system for controlling two sets of emitters with two control signals according to an embodiment of the subject matter described herein. Referring to FIG. 12, an exemplary configuration of the input and output flow is illustrated in which two control signals marked as control signal (A) (1202) and control signal (B) (1204) are received by a logic circuit (1206) and where the logic circuit receives and processes the two control signals and transmits a new set of control signals marked as control signal (C) (1208) which is the data required for controlling the first set of emitters [subset A] (1210) and the first set of emitters [subset B] (1212), control signal (D) (1214) which is received by the modular inner unit and then processed and used to control the attached emitters (1216), and control signal (E) (1218) which (if selected and configured) is sent as an output signal to additional sensory stimulation feedback systems and/or compatible connected devices, systems, or network destinations.

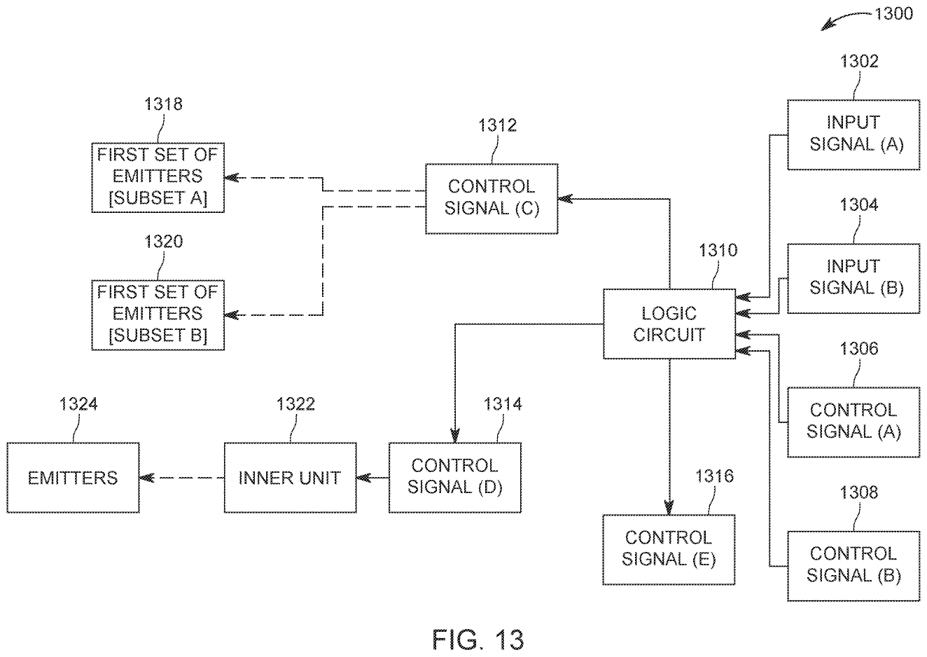

FIG. 13 is a diagram illustrating an exemplary configuration of a sensory stimulation feedback system for controlling two sets of emitters with two input signals and two control signals according to an embodiment of the subject matter described herein. Referring to FIG. 13, an exemplary configuration of the input and output flow is illustrated in which two input signals marked as input signal (A) (1302) and input signal (B) (1304) are received along with two control signals marked as control signal (A) (1306) and control signal (B) (1308) by a logic circuit (1310) and where the logic circuit receives and processes the two input signals and the two control signals and transmits a new set of control signals marked as control signal (C) (1312) which is the data required for controlling the first set of emitters [subset A] (1318) and the first set of emitters [subset B] (1320), and control signal (D) (1314) which is received by the modular inner unit (1322) and then processed and used to control the attached emitters (1324), and control signal (E) (1316) which (if selected and configured) is sent as an output signal to additional sensory stimulation feedback systems and/or compatible connected devices, systems, or network destinations.

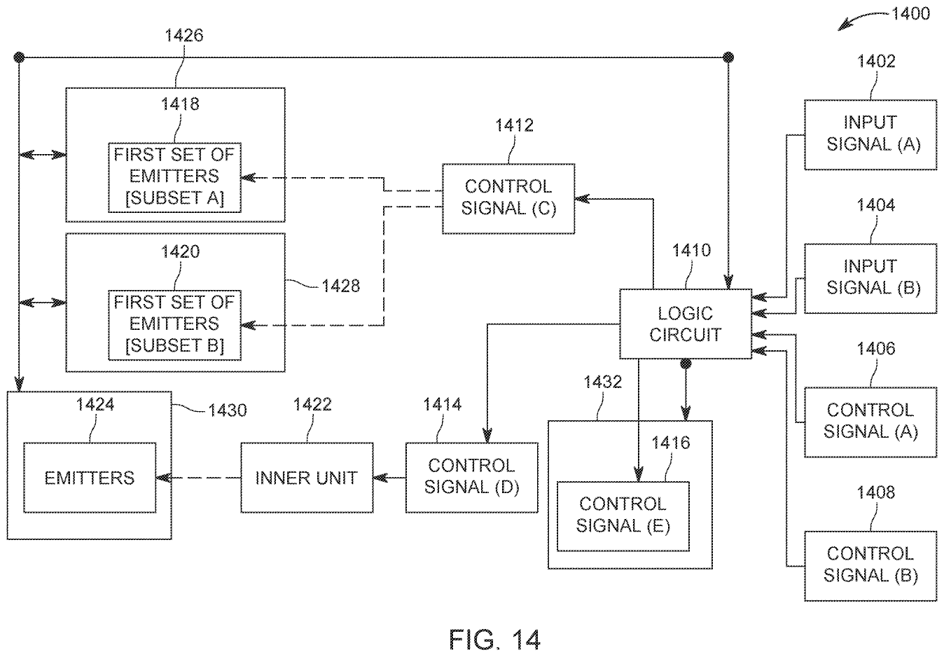

FIG. 14 is a diagram illustrating an exemplary configuration of a sensory stimulation feedback system for controlling two sets of emitters with two input signals and two control signals according to an embodiment of the subject matter described herein. Referring to FIG. 14, an exemplary configuration of the input and output flow is illustrated in which two input signals marked as input signal (A) (1402) and input signal (B) (1404) are received along with two control signals marked as control signal (A) (1406) and control signal (B) (1408) by a logic circuit (1410) and where the logic circuit receives and processes the two input signals and the two control signals and transmits a new set of control signals marked as control signal (C) (1412) which is the data required for controlling the first set of emitters [subset A] (1418) and the first set of emitters [subset B] (1420), and control signal (D) (1414) which is received by the modular inner unit (1422) and then processed and used to control the attached emitters (1424), and control signal (E) (1416) which (if selected and configured) is sent as an output signal to additional sensory stimulation feedback systems and/or compatible connected devices, systems, or network destinations.

Referring still to FIG. 14, the boxes attached to (1426), (1428), (1430), and (1432) represent a bidirectional hardware detection circuit. For each bidirectional hardware detection circuit, the modular attachment or detachment of the set would be detected by the logic circuit (1410) and set as either available or unavailable to the logic circuit (1410).

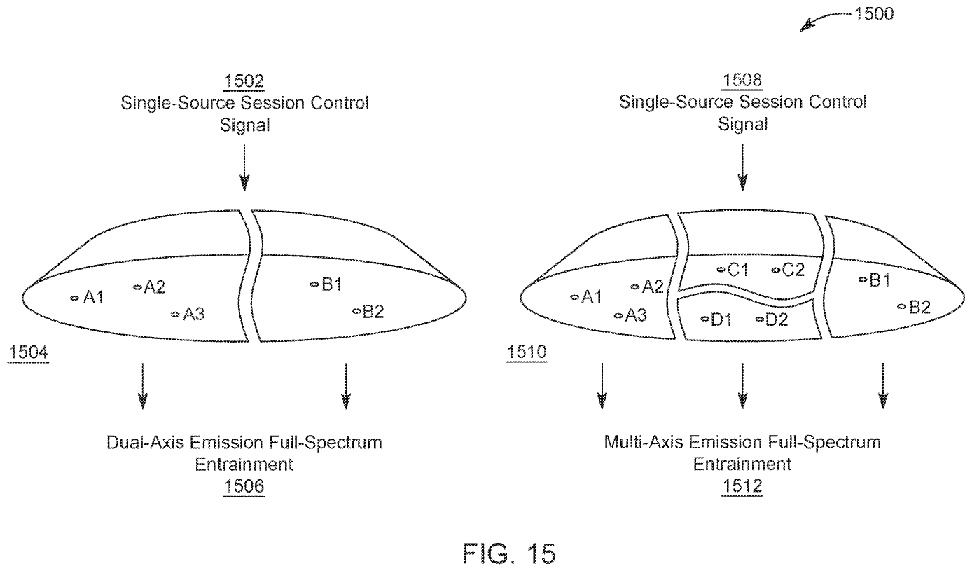

FIG. 15 is an illustration of a software configured dual zone/dual axis configuration for a sensory stimulation feedback system using a single source session control signal for dual axis emission full spectrum entrainment according to an embodiment of the subject matter described herein. Referring to FIG. 15, a single-source session control signal (1502) is shown being transmitted to an array (1504). The array (1506) includes A1, A2, A3, B1, and B2 reference LEDs. The dual axis emission is software configured and controlled to split the array (1506) into two sets of paired energy emitters where a pair would consist of two or more energy emitters interacting together to create an energy wave of a single characteristic (A1, A2, and A3 as one paired set . . . B1 and B2 as another paired set), to emit a dual-axis, full-spectrum entrainment energy session (1506). Such hardware and software configurations provide a fuller multi-optic (multiple light sets), stereo-channel (dual output), multi-chromatic (full-color range), sensory entrainment “session.” When looking at the timeline of existing art and the invention outlined here, this technology is not too dissimilar to that of a high-fidelity stereo sound system in that a single control signal is input and a set of paired energy waves of a high-fidelity characteristic are output from a set of paired transducers/energy emitters.

FIG. 15 is also an illustration of a software configured multi-zone/multi-axis configuration for a sensory stimulation feedback system using a single source session control signal (1508) for multi-axis emission full spectrum entrainment according to an embodiment of the subject matter described herein. Referring to FIG. 15, a single-source session control signal (1508) is shown being transmitted to an array (1510). The array (1510) includes A1, A2, A3, B1, B2, C1, C2, D1, and D2 reference LEDs. The multi-axis emission is software configured and controlled to split the array (1510) into multiple sets of paired energy emitters where a pair would consist of two or more energy emitters interacting together to create an energy wave of a single characteristic (A1, A2, and A3 as one paired set . . . B1 and B2 as another paired set . . . D1 and D2 as yet another paired set . . . and C1 and C2 as yet another paired set), to emit a multi-axis, full-spectrum entrainment energy session (1512). Such hardware and software combinations provide the ideal upper range of capabilities for a multi-optic (multiple light sets), multi-channel (multi output), multi-chromatic (full-color range), sensory entrainment “session.” When looking at the timeline of existing art and the invention outlined here, this technology is not too dissimilar to that of a high-fidelity surround-sound audio system in that a single control signal is input and multiple sets of paired energy waves of a high-fidelity characteristic are output from multiple sets of paired transducers/energy emitters.

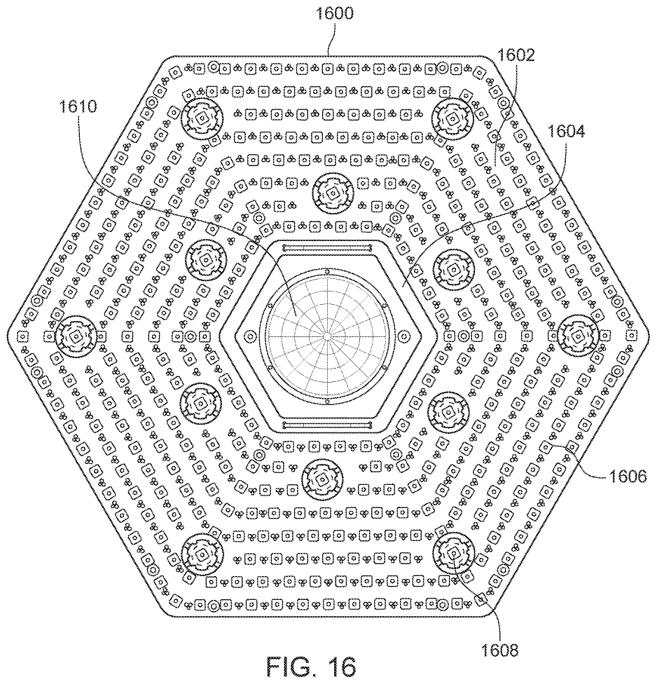

FIG. 16 is an illustration of the front view of a sensory stimulation feedback system with a modular halogen inner unit 1610 according to an embodiment of the subject matter described herein. The sensory stimulation feedback system shown has a hexagonal shape 1600 and a substantially flat front surface. The outer unit of the sensory stimulation feedback system includes a plurality of surface mounted LEDS 1602 and 1606 but may include other emitters or no emitters. The LEDs may be arranged in one or more strips, lines, rows, columns or other physical configurations. The LEDs may also be connected to a power source and the controller individually or in one or more strips, lines, rows, columns, etc. The sensory stimulation feedback system shown also includes several small screw holes 1608 for mounting various types of attachments (e.g., diffusion layers). The 12 circular holes 1608 shown symmetrically distributed in the outer unit, allow for the emissions of the 12 individually controlled high powered RGBW LEDs (see FIG. 16) to emit without obstruction.

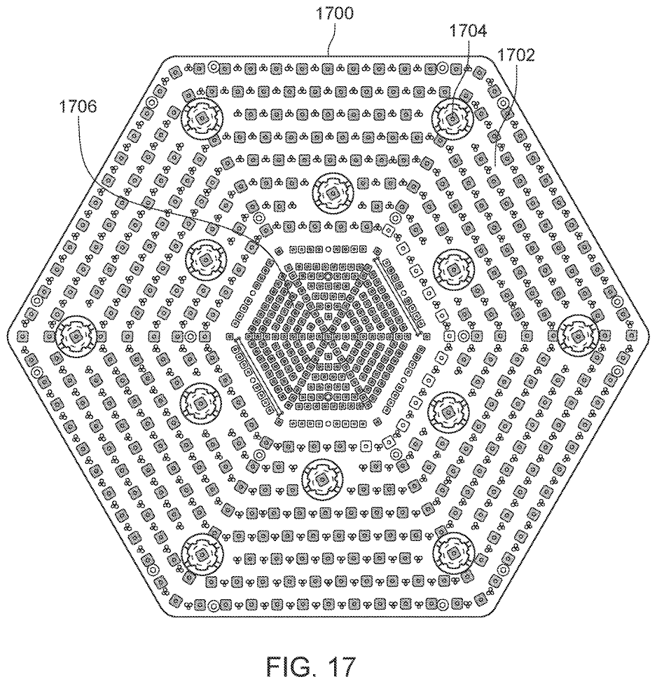

FIG. 17 is an illustration of the front view of a sensory stimulation feedback system with a modular high-density LED inner unit, containing 470 surface mounted LEDs 1706 while powered on according to an embodiment of the subject matter described herein. The sensory stimulation feedback system when powered on may emit light from any or all of the emitters. As discussed above, operation of the emitters 1702 may be controlled individually or in groups. This may include turning an emitter on or off for a period of time to produce solid/continuous light or flashing/strobing light at a desired frequency. This may also include controlling the intensity, color, or other parameters of the emitters over time. Here, the sensory stimulation feedback system includes a modular high-density LED inner unit, displaying a pattern of varying intensities and colors, and an outer unit displaying a second pattern of a constant intensity and color that is different from the first pattern. Here it can be observed that while the first set of emitters [subset A] 1702 and 1706 and the first set of emitters [subset B] 1704 are displaying the same color, the intensity differs.

FIG. 18 is an illustration of a front view of a sensory stimulation feedback system 1700 with a modular high-density LED inner unit when powered off according to an embodiment of the subject matter described herein. Here, it may be appreciated that the size of each of the 470 emitters 1802 in the modular inner unit is smaller than both the small LEDs and the high-power emitters of the outer unit. Additionally, it may be appreciated that the density of the emitters in the modular inner unit is higher than that of either the small LEDs or the high-power emitters in the outer unit.

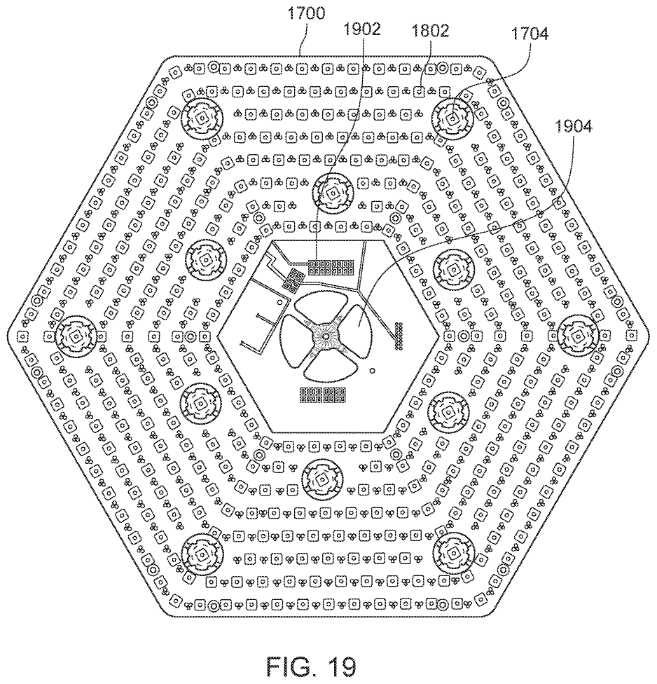

FIG. 19 is an illustration of a front view of a sensory stimulation feedback system without a modular inner unit according to an embodiment of the subject matter described herein. Here, the communications and power connector(s) 1902 and 1904 may be seen. Corresponding communications and/or power connector(s) may be located on the rear side of the modular inner unit (FIG. 40) such that the modular inner unit may be detachably coupled to the system. Additional means for physically connecting or securing the modular inner unit to the outer 1902 unit may also be used, or the communications and/or power connector(s) may provide sufficient coupling.

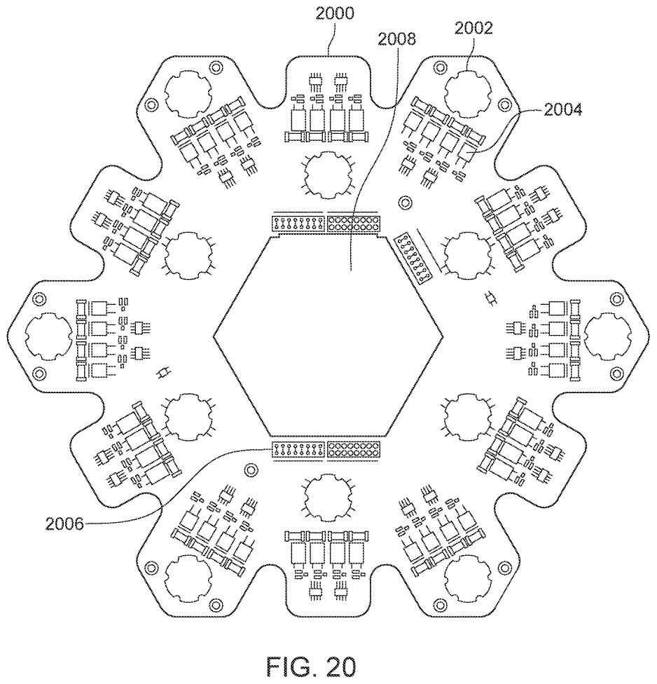

FIG. 20 is an illustration of the rear view of a high-power LED light module of a sensory stimulation feedback system according to an embodiment of the subject matter described herein. Here, a printed circuit board (PCB) 2000 may include a plurality of locations 2002 associated with the set of high-power RGBW LEDs discussed above surrounding a central hexagonal opening 2008 associated with accepting a modular inner unit. Each of the locations may include associated circuitry 2004 for individually powering and controlling the operation of each of the high-power LEDs. This PCB may be located between the front surface of the outer unit of a sensory stimulation feedback system and a rear housing or other components.

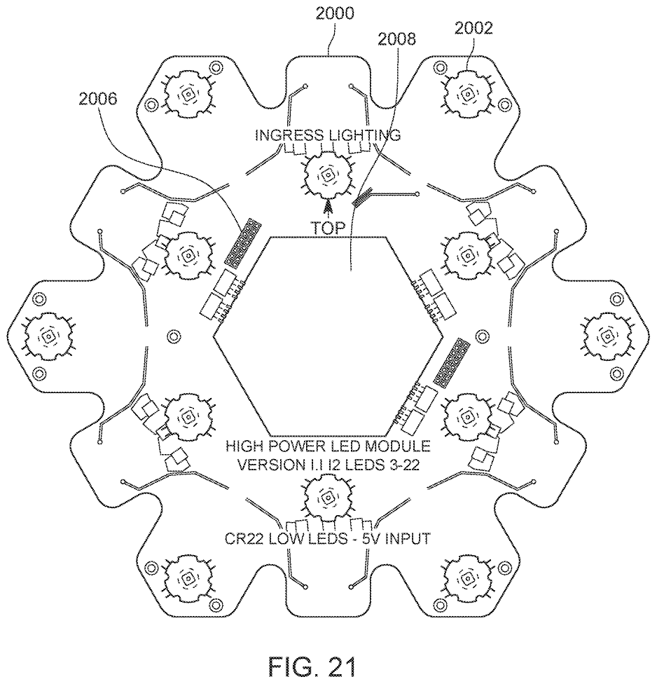

FIG. 21 is an illustration of a front view of a high-power LED light module of a sensory stimulation feedback system 2000 according to an embodiment of the subject matter described herein. Here, each of the twelve (12) high power RGBW LEDs 2002 are shown on a front-facing portion of the PCB shown in FIG. 20. Additionally, two connectors 2006 are shown for integrating the PCB with other components of the sensory stimulation feedback system.

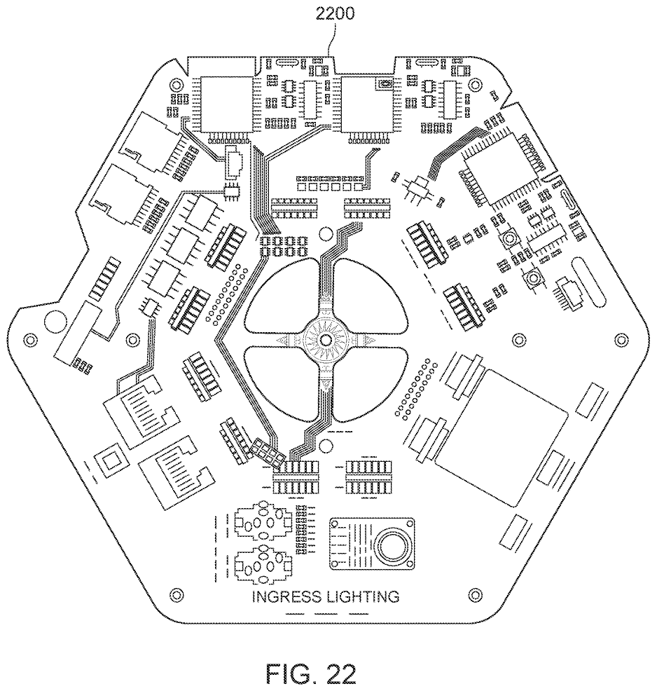

FIG. 22 is an illustration of the rear view of a controller module of a sensory stimulation feedback system 2200 according to an embodiment of the subject matter described herein. Here, various circuitry, memory, processors, electrical connections, and network interfaces may be included on a controller. Input and/or control signals may be received and processed by the controller shown for controlling the operation of associated emitters in a sensory stimulation feedback system. External communications may be performed using any suitable communications standard.

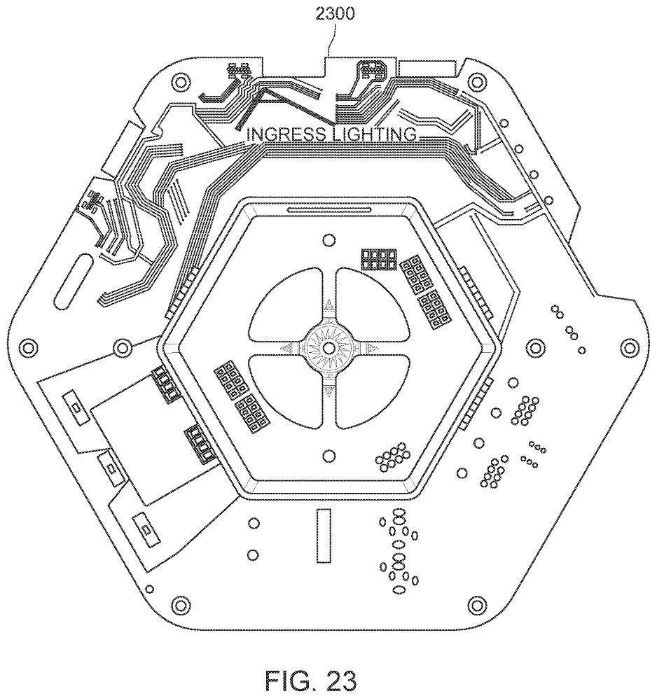

FIG. 23 is an illustration of the front view of a processing module of a sensory stimulation feedback system 2300 according to an embodiment of the subject matter described herein. Here, connectors are shown for integrating the PCB with other components of the sensory stimulation feedback system. For example, two of the connectors shown may correspond to the two connectors of the high-power LED light module shown in FIG. 21.

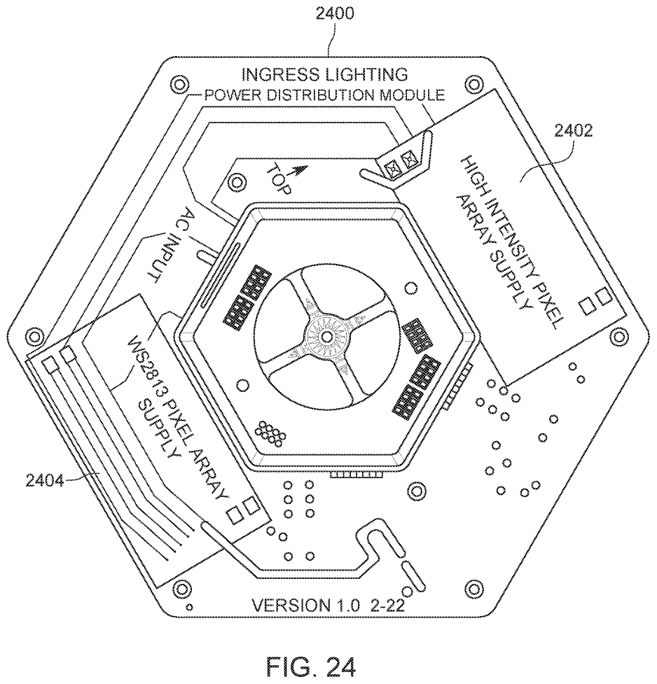

FIG. 24 is an illustration of the rear view of a power distribution module of a sensory stimulation feedback system 2400 according to an embodiment of the subject matter described herein. Here, two of the power supplies (the “high intensity pixel array supply” 2402 and the “WS2813 pixel array supply” 2404 which are connected on the other side) are labeled. The AC power input location is also documented.

The high intensity pixel array supply may be connected to a power source for supplying power to the twelve high intensity RGBW LEDs shown in FIGS. 17 and 21. This may include a battery, if the power source is an internal power source, or may include circuitry for receiving power from an external power source, such as voltage regulation, AC-to-DC conversion circuitry, etc.

The WS2813 pixel array supply may also be connected to a power source for supplying power to a WS2813 addressable RGB LED module. In one embodiment, the WS2813 addressable RGB LED module includes a DC5V built-in integrated circuit (IC) featuring break-point continuous transmission signal. The WS2813 integrates the control circuit and the RGB chip into a component package and includes an internal oscillator to achieve a highly consistent color effect.

The protocol for communicating data with a WS2813 addressable RGB LED may be a single NZR communication mode. For example, a DIN port on the WS2813 may receive data from the controller. After pixel power-on or reset, a first pixel may collect initial 24-bit data that is sent to an internal data latch. Other data, which may be reshaped by an internal signal reshaping amplification circuit, may be sent to the next cascade pixel via a DO port. After transmission, the signal bit depth may be decreased for each pixel. Each WS2813 LED pixel may also adopt auto-reshaping transmission technology such that the pixel cascade numbers are not limited to the transmission of the signal. Further, a BIN may receive a data signal and compare the data to the DIN side. If the DIN does not receive the signal, the DIN may then switch to BIN for receiving the input signal to ensure that any damage to the IC does not affect signal cascade transmission.

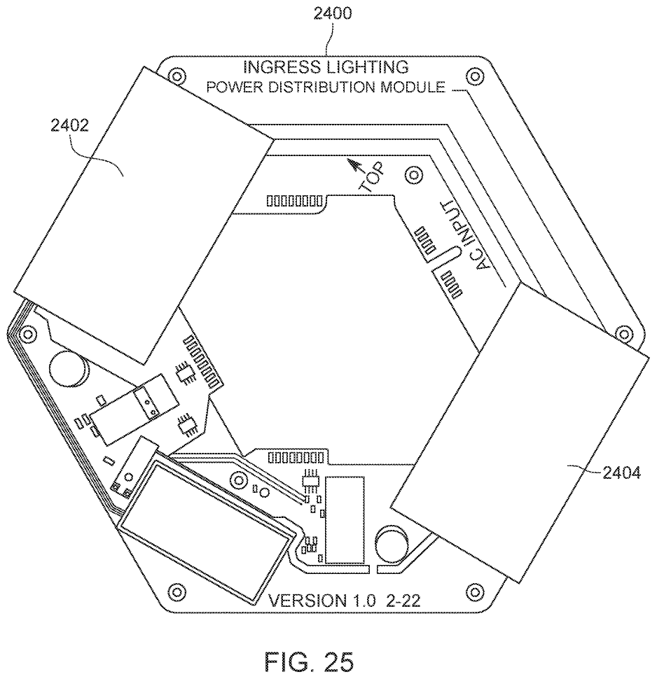

FIG. 25 is an illustration of the front view of a power distribution module of a sensory stimulation feedback system according to an embodiment of the subject matter described herein. Here, multiple power supplies are attached and viewed as contained within rectangular housings corresponding to the locations shown in FIG. 24, including a 5 volt smaller power supply along with the WS2813 pixel array supply and the high intensity pixel array supply modules.

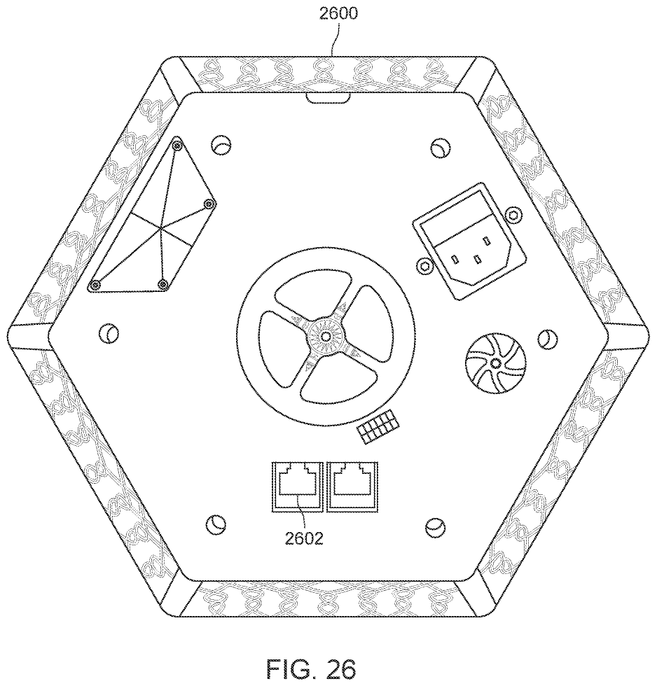

FIG. 26 is an illustration of the rear view of an outer housing of a sensory stimulation feedback system according to an embodiment of the subject matter described herein. Here, the outer housing hides most of the internal components of the sensory stimulation feedback system. This illustration shows the two dedicated DMX ethernet ports, located and accessible for communicating with other DMX devices. The metal touch sensor is visible which is located on the rear of the unit as seen in this figure which can be used to control the sensory stimulation feedback system. The AC power input jack can also be seen here. The cutout which allows for the microphone to be unobstructed can be seen between the DMX ethernet ports and the AC power jack. The screw holes in FIG. 21 are in place to accommodate a variety of accessories as well as system cooling options.

FIG. 27 is an illustration of the side view of an outer housing of a sensory stimulation feedback system according to an embodiment of the subject matter described herein. Here, the outer housing substantially matches and wraps around the hexagonal shape of the front face of the sensory stimulation feedback system and tapers down to match the smaller shape of the rear housing. Additionally, replaceable side panels constructed from PCB can be seen. One or more perforations in the housing and side panels may be used for ventilation and/or sound transmission in some embodiments.

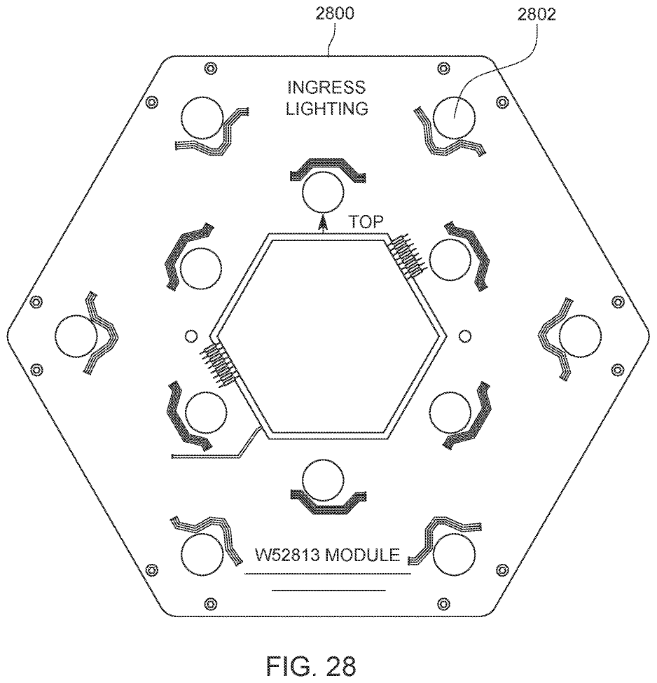

FIG. 28 is an illustration of a back view of a WS2813 addressable RGB LED module of a sensory stimulation feedback system according to an embodiment of the subject matter described herein. Here, connectors for supplying power and data to the board and through holes for the twelve high intensity RGBW LEDs can be seen.

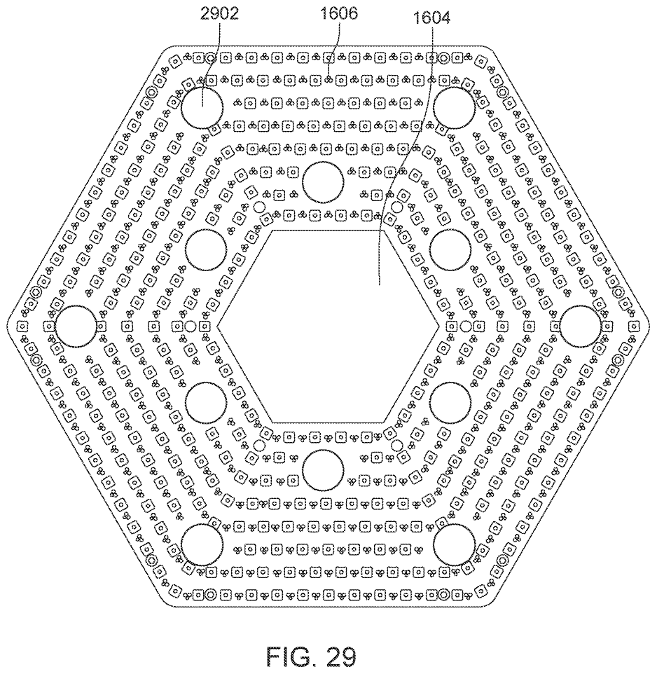

FIG. 29 is an illustration of a front view of a WS2813 addressable RGB LED module of a sensory stimulation feedback system according to an embodiment of the subject matter described herein. Here, 372 small RGB LEDs are shown mounted to the front surface of the PCB in a pixel array/first set of emitters. As mentioned above, each of the 372 small RGB LEDs may be controlled by the WS2813 addressable RGB LED module individually or in groups, strips, zones, etc. For example, all LEDs located in a first quadrant may be activated together. Alternatively, all LEDs in the outermost ring may be activated together, and so on in various combinations.

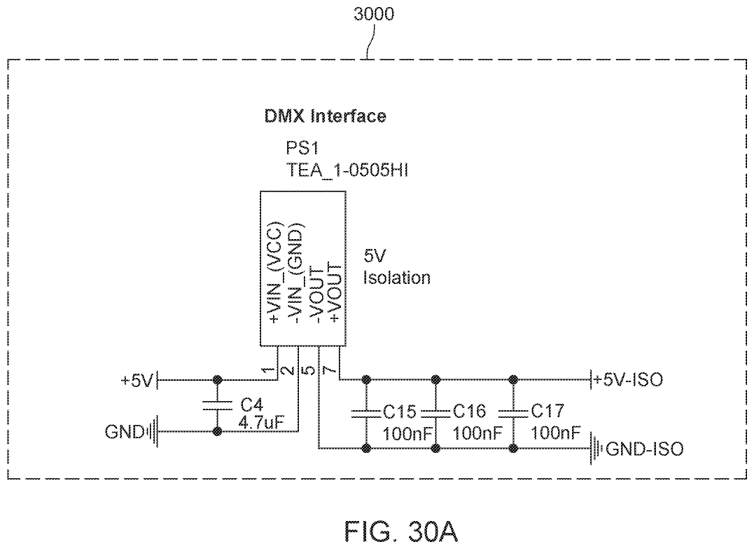

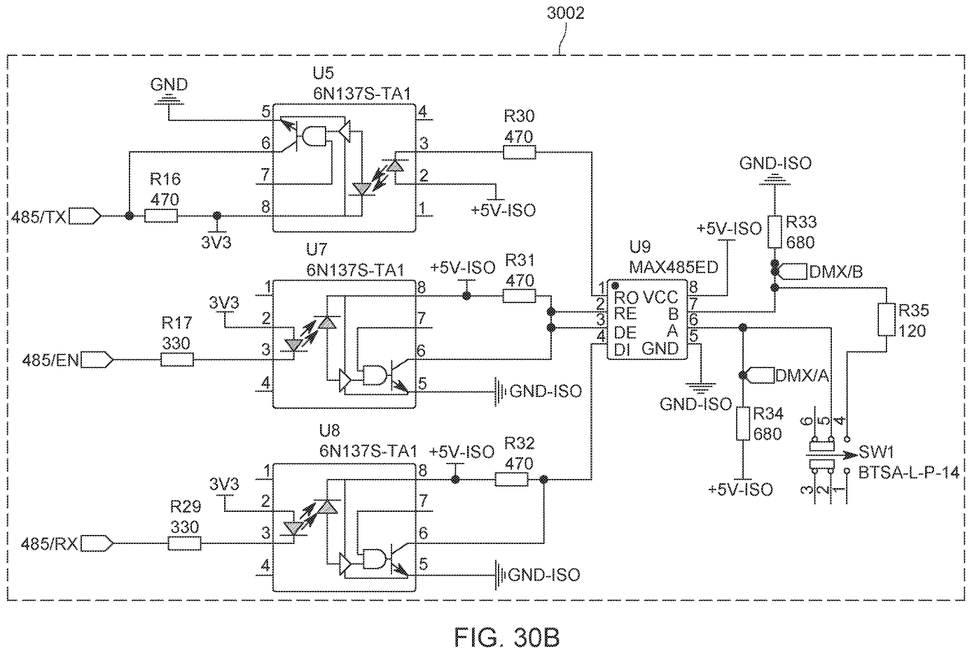

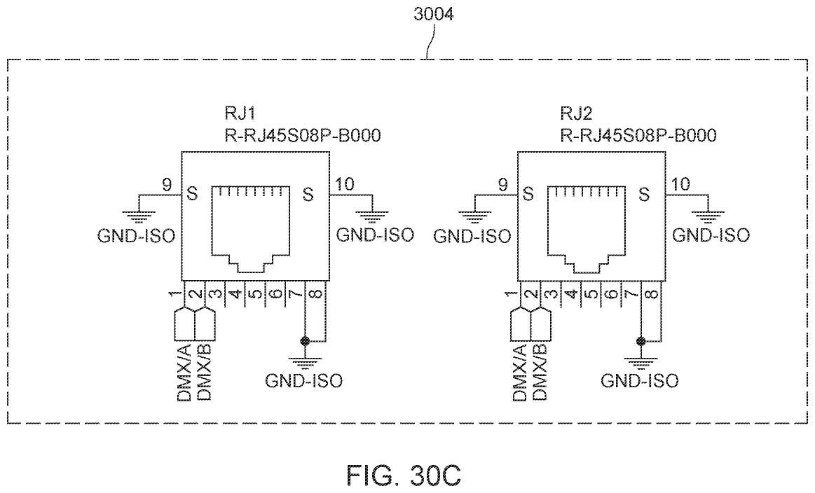

FIGS. 30A, 30B, and 30C are a schematic diagram of a DMX interface component of a sensory stimulation feedback system according to an embodiment of the subject matter described herein. DMX512 (DMX) is a standard for digital communication networks used to control lighting and effects. DMX512 uses a unidirectional EIA-485 (RS-485) differential signaling at its physical layer, in conjunction with a variable-size, packet-based communication protocol. DMX512 does not include automatic error checking and correction.

A DMX512 network employs a multi-drop bus topology with nodes strung together in a daisy chain. A network consists of a single DMX512 controller—which is the master of the network—and one or more slave devices. For example, a computing device (e.g., a lighting console or a sensory stimulation feedback system) may act as the controller for a network of slave devices such as intelligent lights/sensory stimulation feedback systems. Each slave device may have a DMX512 “IN” connector and an “OUT” (or “THRU”) connector. The controller, which may only have an OUT connector, may be connected via a DMX512 cable to the IN connector of the first slave. A second cable then links the OUT or THRU connector of the first slave to the IN connector of the next slave in the chain, and so on.

A DMX512 network is called a “DMX universe”. Each OUT connector on a DMX512 controller can control a single universe. A DMX512 universe is made up of 512 channels, with each channel containing a value between 0 and 255. Each slave device in the chain can “look at” a different set of channels in order to be controlled by the master controller. Smaller controllers may have a single OUT connector, enabling them to control only one universe, whereas large controllers may have the capacity to control multiple universes, with an OUT connector provided for each universe. Some controllers, instead of featuring multiple OUT connectors, may have an Unshielded Twisted Pair connector (e.g., CAT5, CAT5e or CAT6 ethernet).

Wireless DMX512 adapters may also be used. A wireless DMX512 network employs a wireless transmitter at the controller and receiver(s) to convert the wireless signal back to conventional DMX512 wired network signals or wireless receivers built into the individual fixtures. Although wireless DMX512 networks can function over distances exceeding 3,000 feet (910 m) under ideal conditions, most wireless DMX512 links are limited to a maximum distance of 1,000-1,500 feet (300-460 m) to ensure reliable operation.

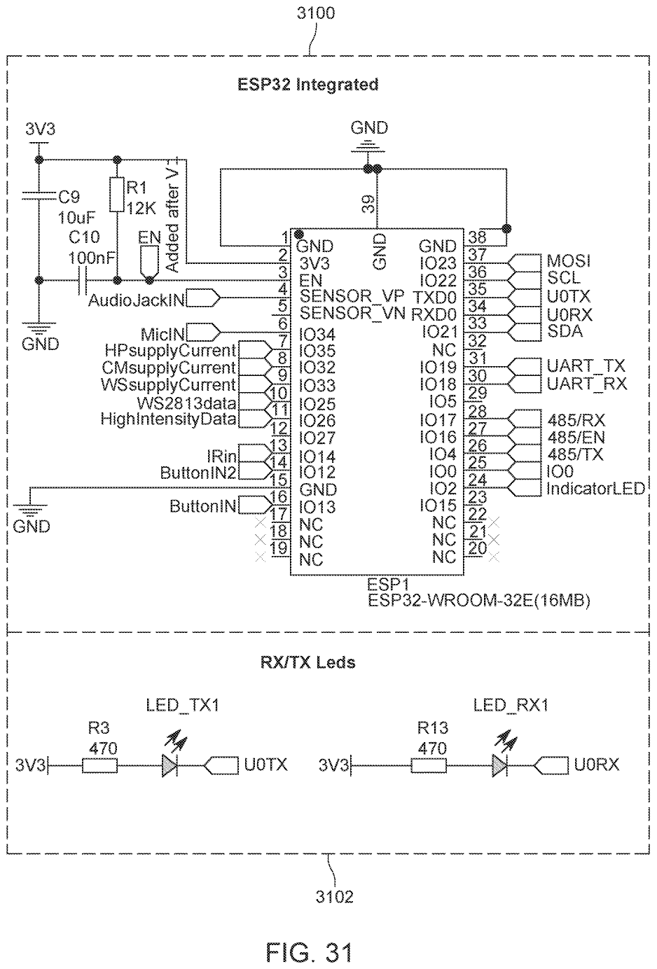

FIG. 31 is a schematic diagram of integrated ESP32 and RX/TX LED components of a sensory stimulation feedback system according to an embodiment of the subject matter described herein. ESP32 is a series of low-power system on a chip microcontrollers with integrated Wi-Fi and dual-mode Bluetooth. The ESP32 series employs either a Tensilica Xtensa LX6 microprocessor in both dual-core and single-core variations, Xtensa LX7 dual-core microprocessor or a single-core RISC-V microprocessor and includes built-in antenna switches, RF balun, power amplifier, low-noise receive amplifier, filters, and power-management modules.

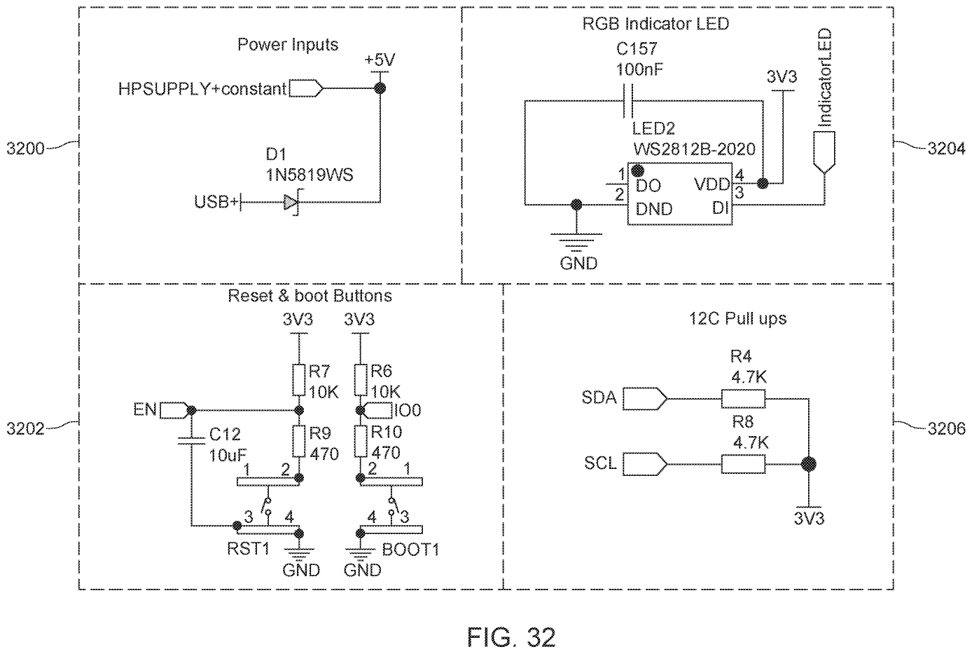

FIG. 32 is a schematic diagram of the power input, RGB indicator LED, Reset and boot button, and I2C pullup components of a sensory stimulation feedback system according to an embodiment of the subject matter described herein. An I2C pullup is an open-drain/open-collector communication standard which supplies integrated circuits with different voltage supply rails which can be connected for communication.

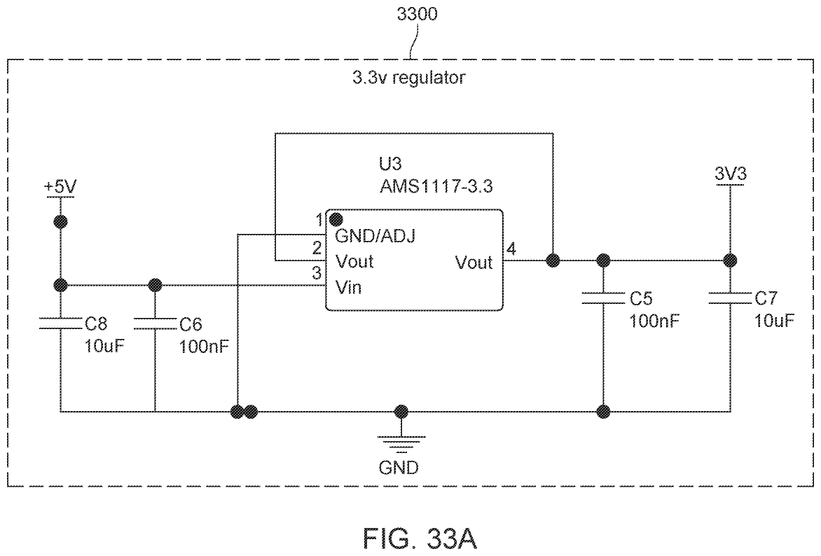

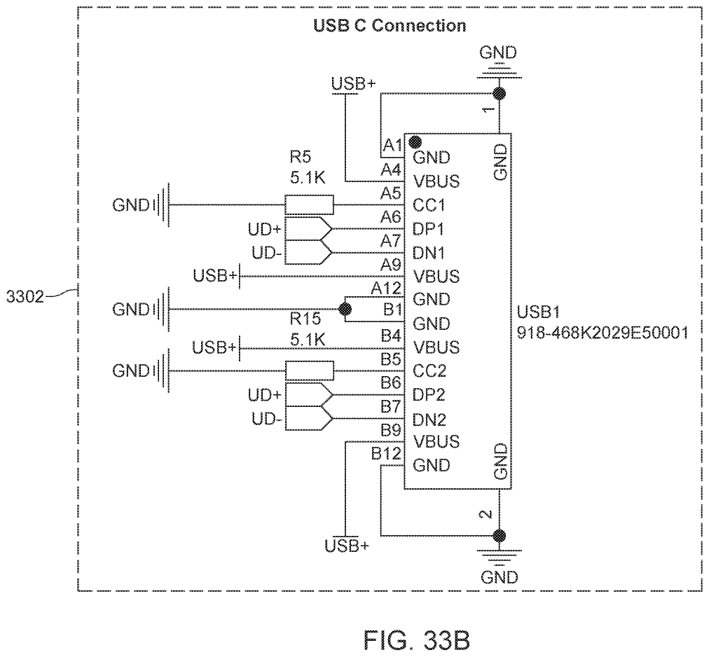

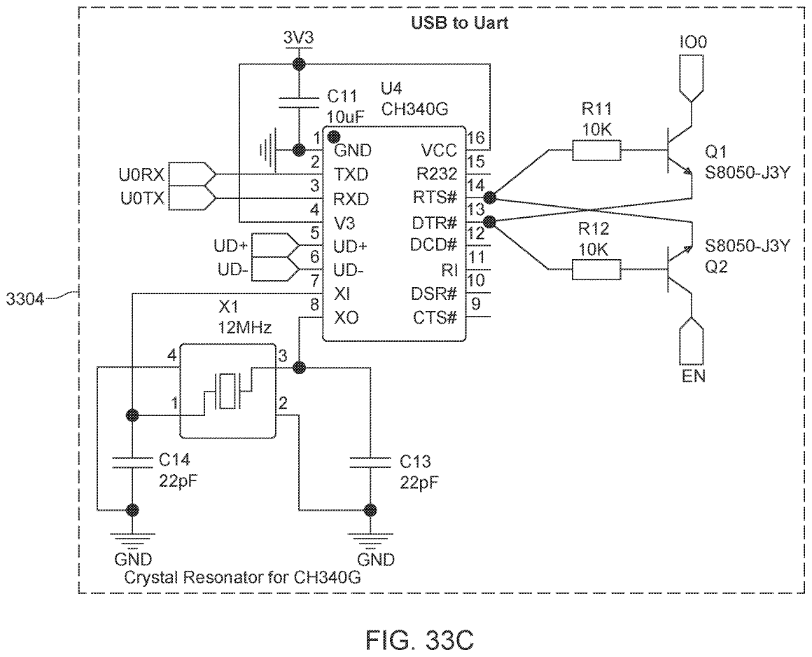

FIG. 33 is a schematic diagram of the voltage regulator, USB type C connector, and USB-to-UART components of a sensory stimulation feedback system according to an embodiment of the subject matter described herein. 3.3V has a lower power consumption but many microcontrollers will not operate correctly at 3.3V as at 5V. Many modules and ICs still target 5V systems and 5V is a defacto power standard due to the abundance of USB power supplies. Therefore, conversion between 5V and 3.3V may be required.

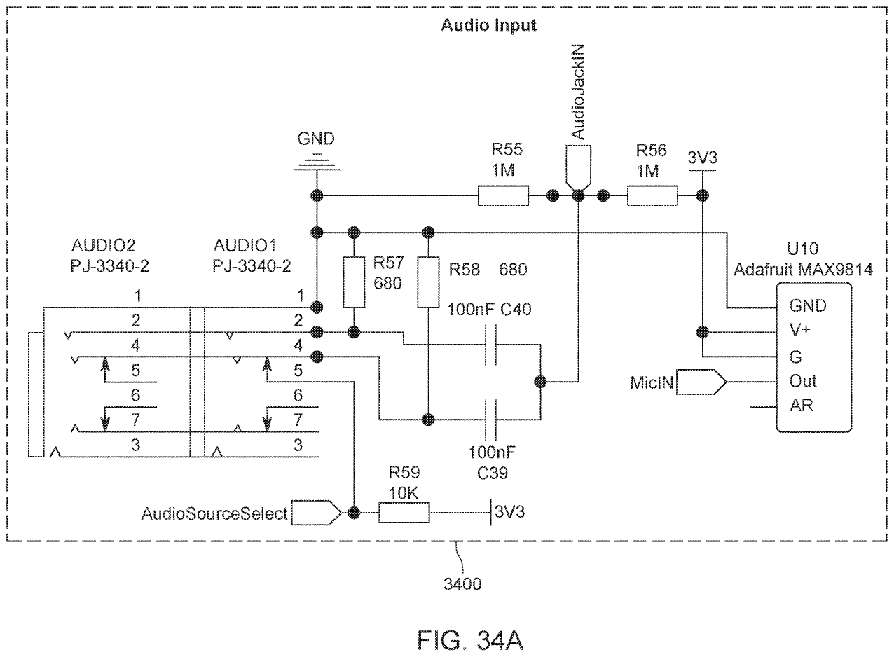

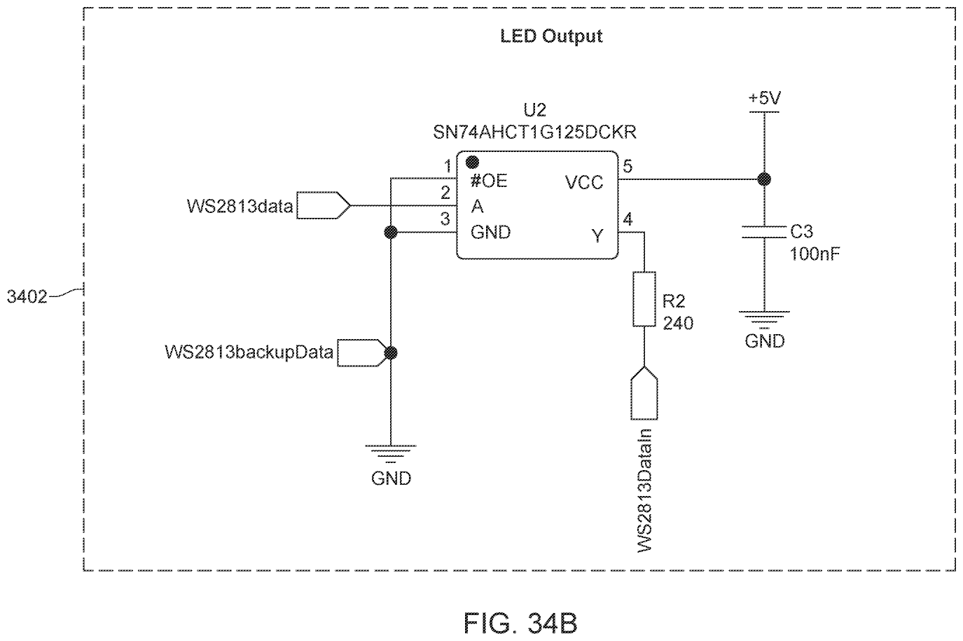

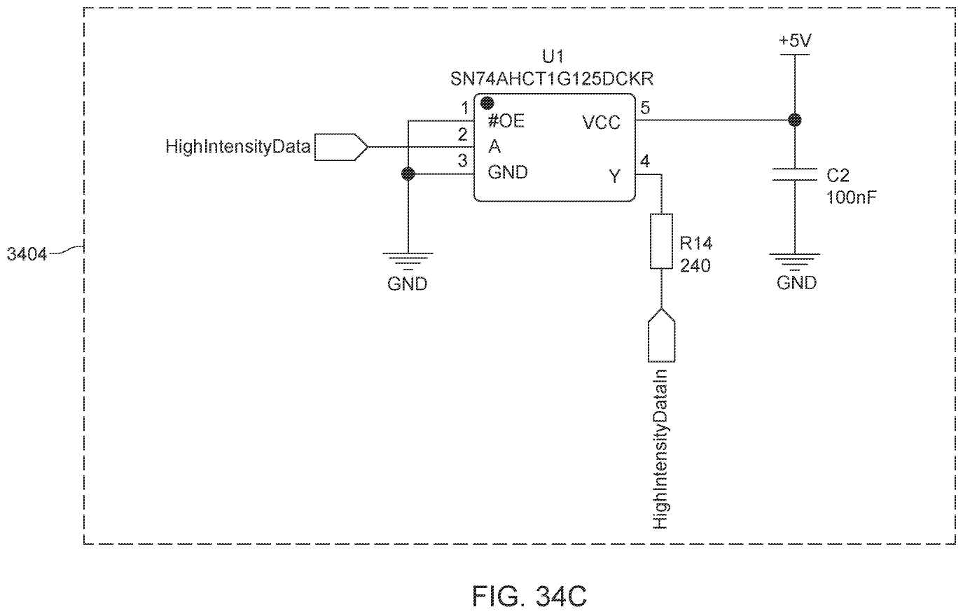

FIG. 34 is a schematic diagram of an audio input component of a sensory stimulation feedback system according to an embodiment of the subject matter described herein. Here, an optional audio input circuit is shown for receiving an audio signal. The audio signal may be passed to an integrated or associated speaker or a set of headphones for providing sound to accompany the sensory emissions emitted by the system. Alternatively, the audio signal may be used as an input source for controlling the operation of the sensory emitters. In this embodiment, the sensory stimulation feedback system may be programmed to synchronize the displayed light pattern(s) or sensory experiences with a received music track or live audio signal. For example, loud bass in the audio signal may be associated with producing a specific intensity of light (e.g., high), a specific color (e.g., red), a strobe effect (e.g., flashing), or any combination thereof.

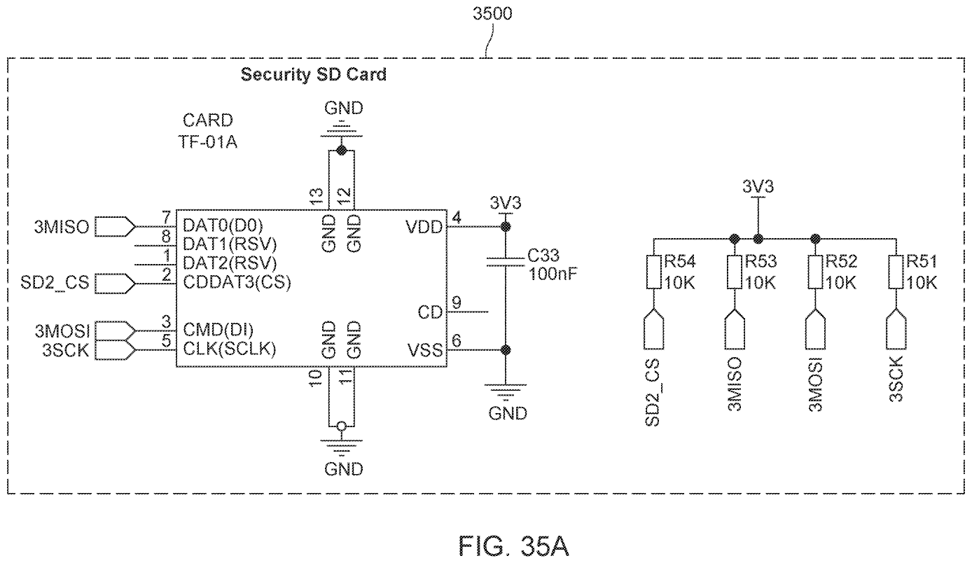

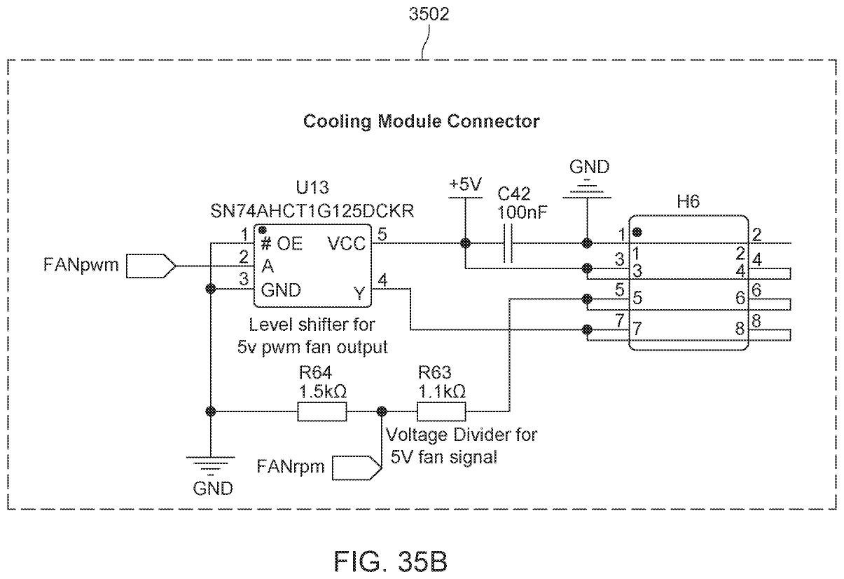

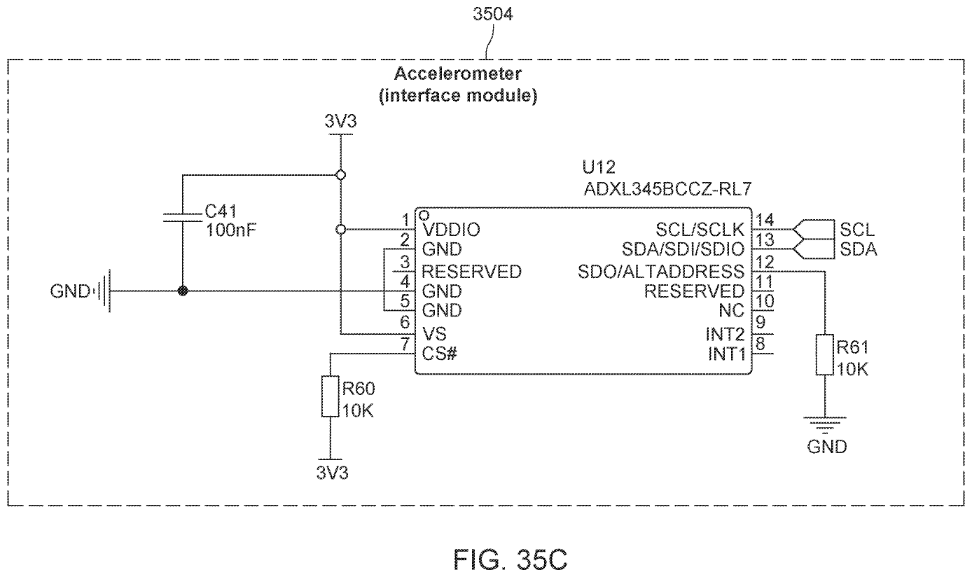

FIG. 35 is a schematic diagram of the security SD card, cooling module connector, and accelerometer interface module components of a sensory stimulation feedback system according to an embodiment of the subject matter described herein. Here, the SD card module may be used for securely reading and storing data on a physical, removable device. This may allow an operator to verify or authenticate themselves for controlling the system, to access the online marketplace, or to activate specific features in the software.

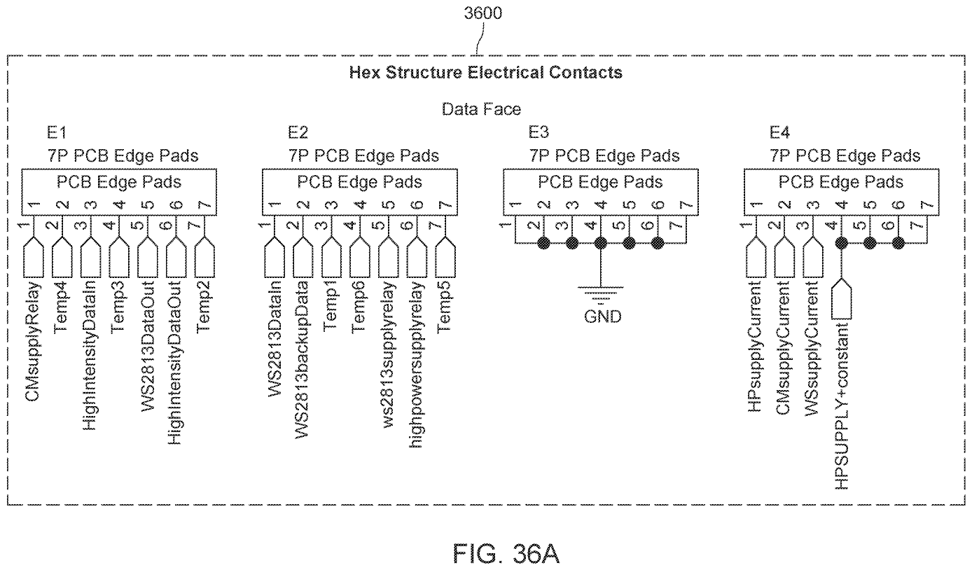

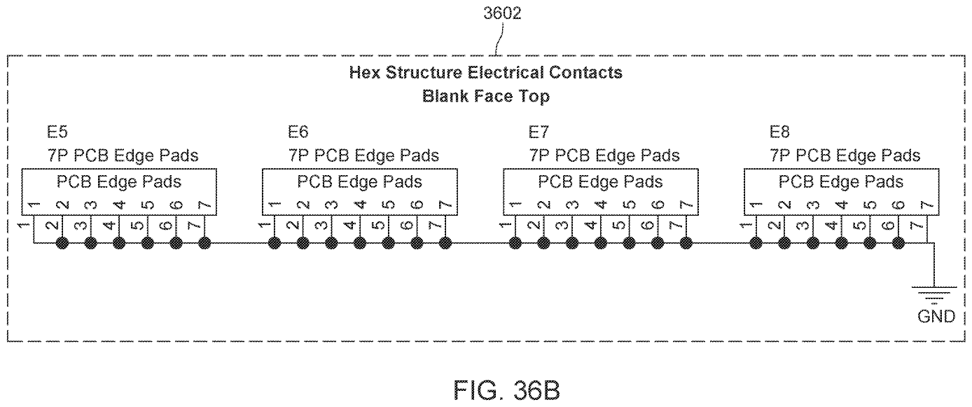

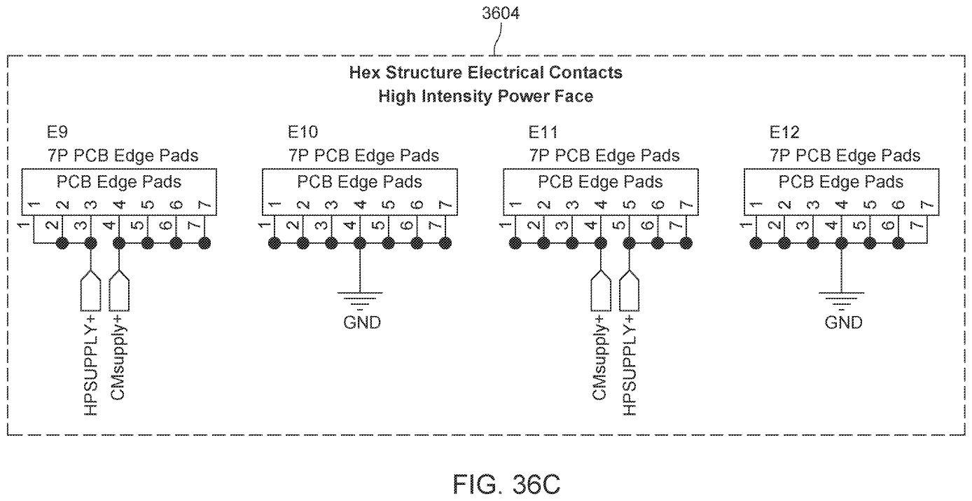

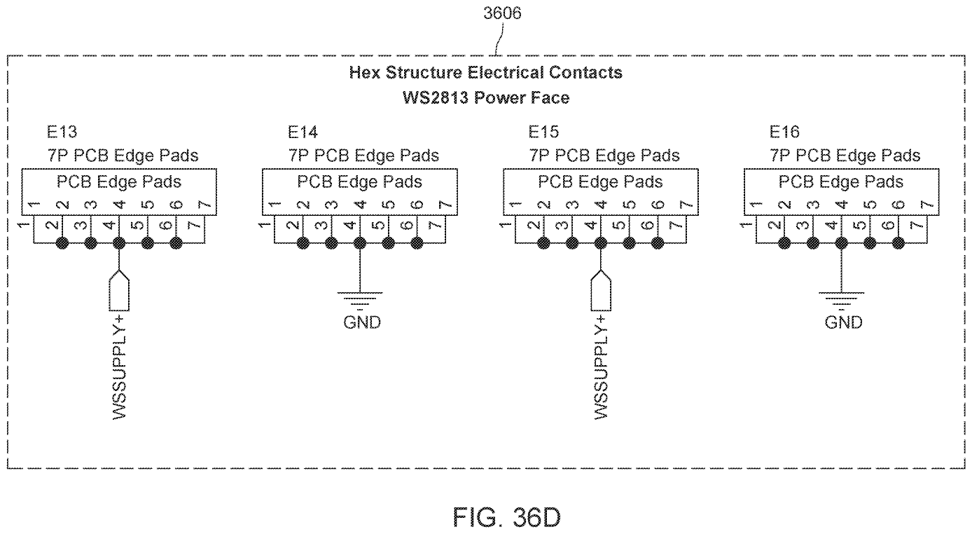

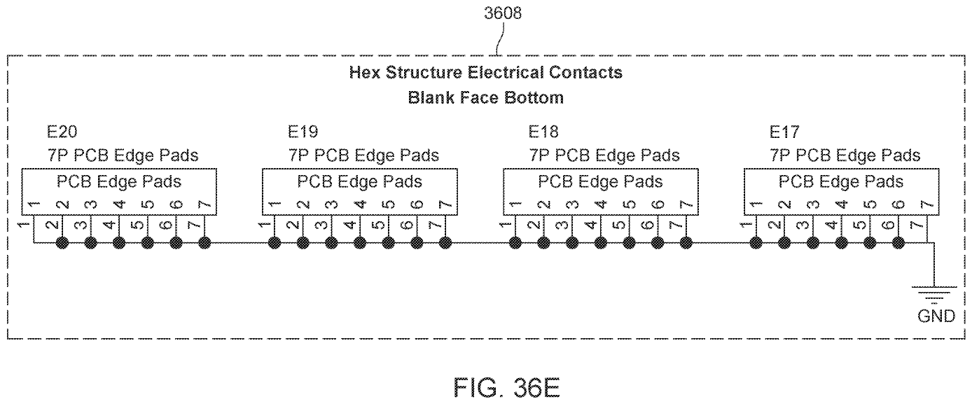

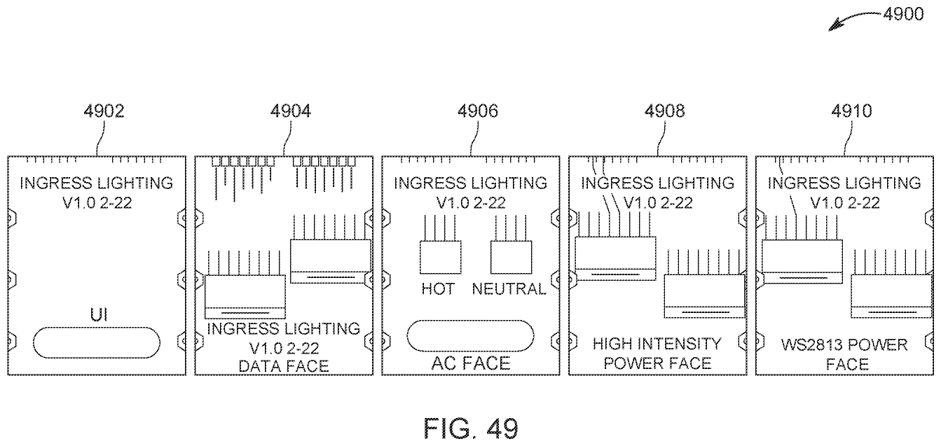

FIG. 36 is a schematic diagram of an internal electrical routing distribution hexagon structure of a sensory stimulation feedback system according to an embodiment of the subject matter described herein. The different hex faces (FIG. 49) route power and data internally, removing the need for wiring.

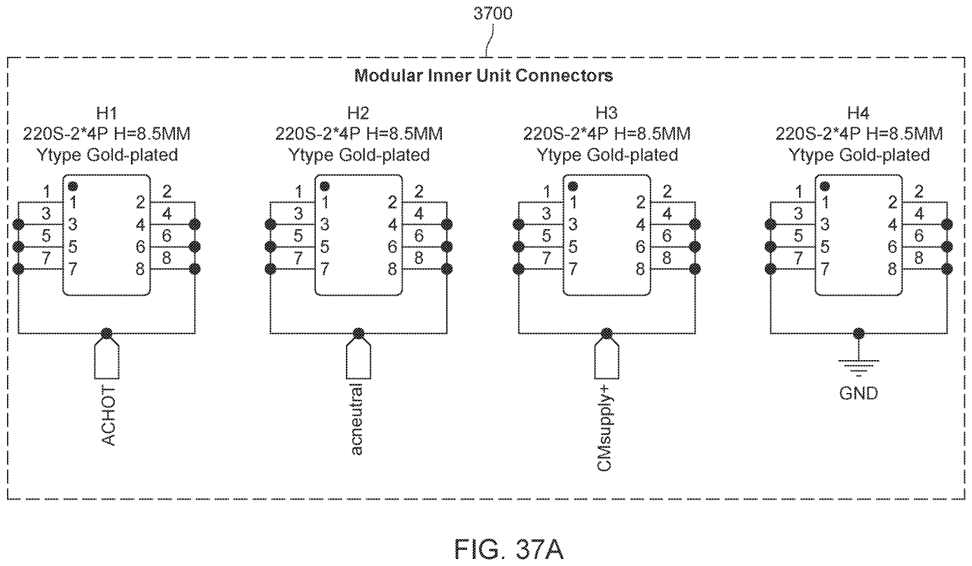

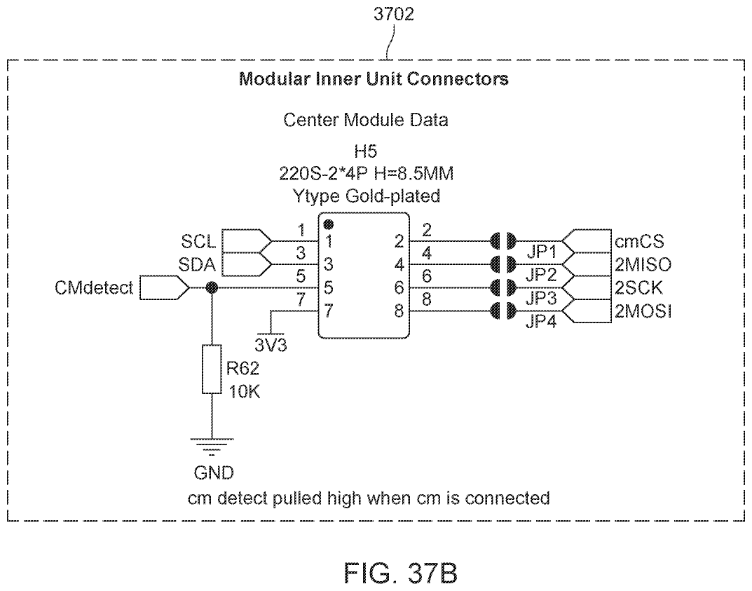

FIG. 37 is a schematic diagram of the modular inner unit connection components of a modular inner unit for communicating and receiving power from a sensory stimulation feedback system according to an embodiment of the subject matter described herein.

FIG. 38 is an illustration of the front view of a modular high density inner unit associated with a sensory stimulation feedback system according to an embodiment of the subject matter described herein. Here, the 470 small RGB LED array can be seen.

FIG. 39 is an illustration of the front view of a modular Halogen inner unit associated with a sensory stimulation feedback system according to an embodiment of the subject matter described herein. Here, the installed Halogen bulb can be seen.

FIG. 40 is an illustration of the front and rear views of a universal modular inner unit associated with a sensory stimulation feedback system according to an embodiment of the subject matter described herein. Here, connectors, components, and circuitry can be seen.

FIG. 41 is an illustration of the front view of a sensory stimulation feedback system with installed LED lenses for the First Set of Emitter(s) [Subset A] and a diffusion layer without a modular inner unit according to an embodiment of the subject matter described herein.

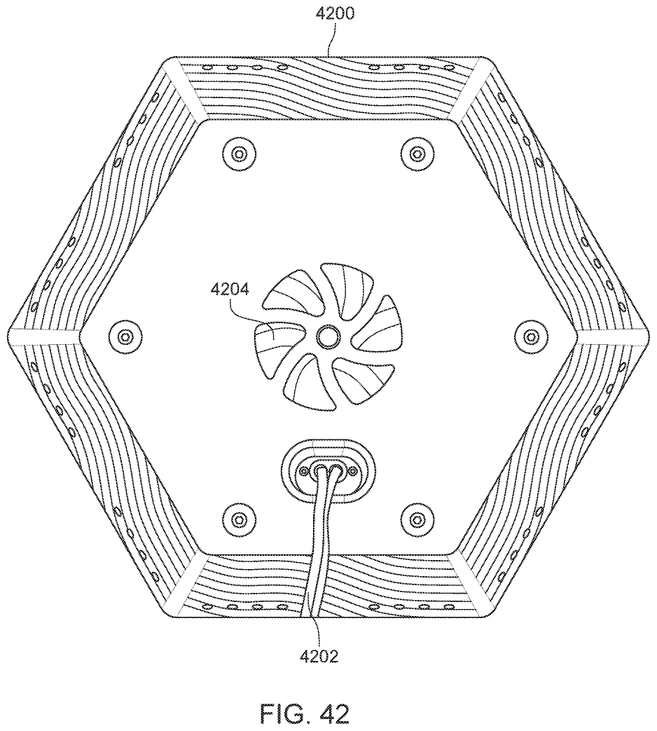

FIG. 42 is an illustration of the rear view of a sensory stimulation feedback system with an installed cooling module according to an embodiment of the subject matter described herein.

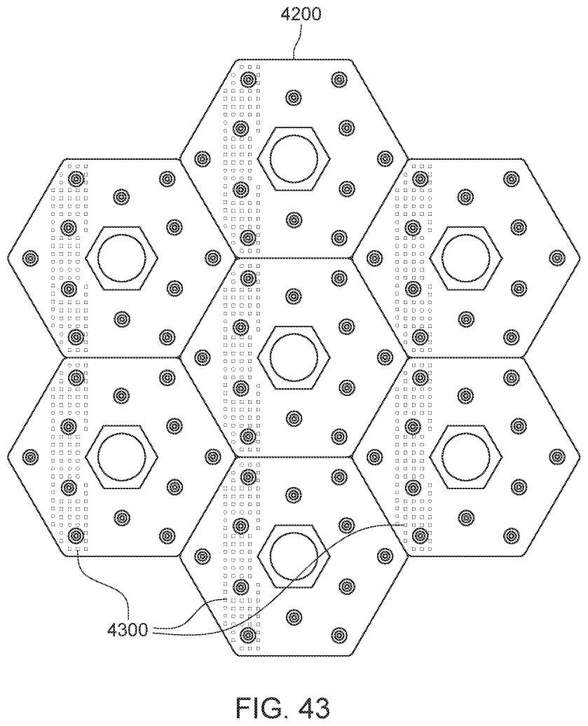

FIG. 43 is a representation of seven separate sensory stimulation feedback system aligned in a matrix where all of the controllers are configured in the same way and are all receiving the same control signal independently which results in all seven systems activating the same emitters, producing the same effect according to an embodiment of the subject matter described herein.

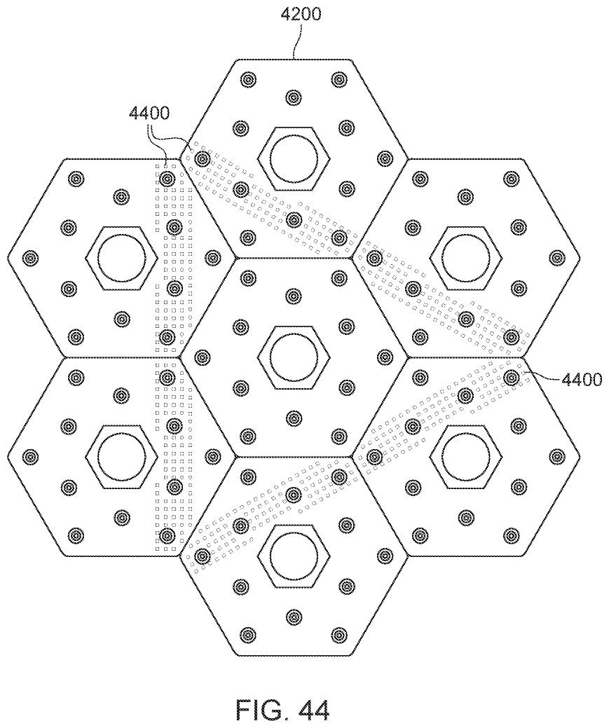

FIG. 44 is a representation of seven separate sensory stimulation feedback system aligned in a matrix where all of the controllers are configured in the same way but are all receiving different control signals independently which results in all seven lights activating different individual emitters, producing a different effect on each system but creating a shared pattern within the matrix according to an embodiment of the subject matter described herein.

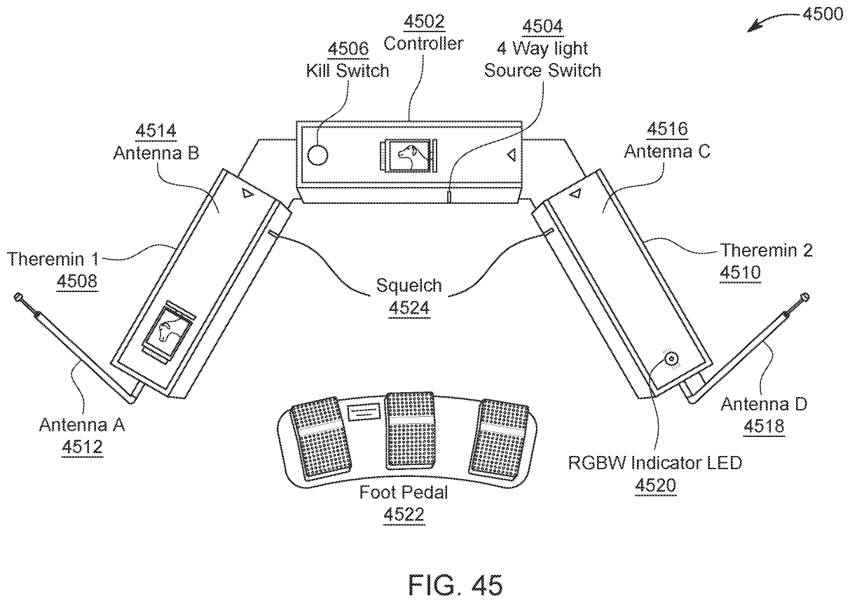

FIG. 45 is a representation of a dual theremin controller, designed to control specific mapped variables of a sensory stimulation feedback system according to an embodiment of the subject matter described herein. Here, system 4500 includes a controller 4502 having a four-way light source switch 4504 and a kill switch 4506. A first theremin 4508 and a second theremin 4510 are associated with antennas A, B 4512, 4514 and antennas C, D 4516, 4518, respectively for communicating with components of the system. An RGBW (red green blue white) LED indicator 4520 is located on theremin 4510. Foot pedal 4522 can be used to control the system. Finally, a squelch 4524 is associated with theremins 4508 and 4510.

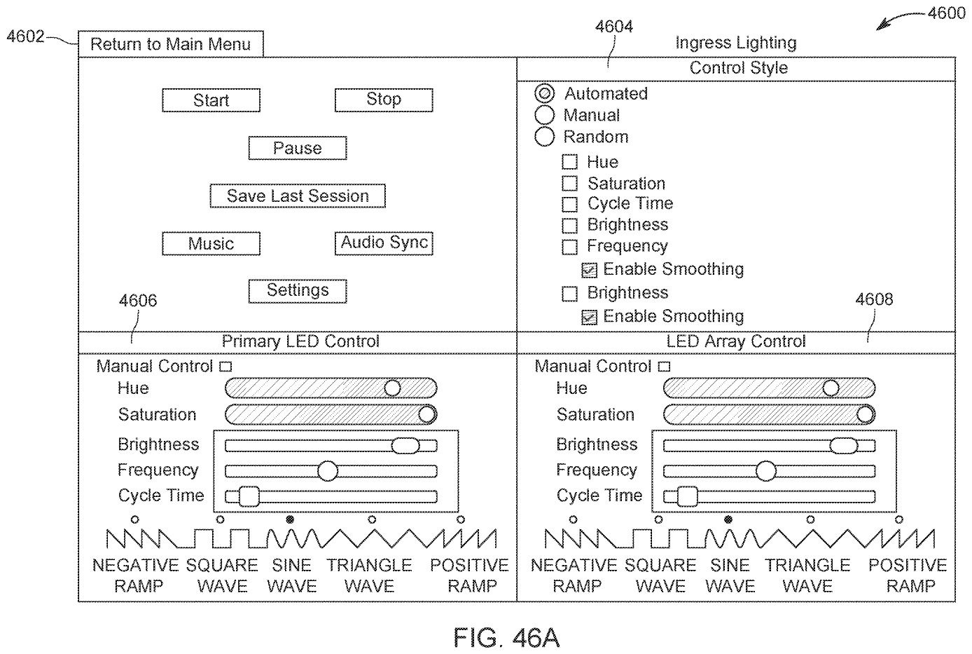

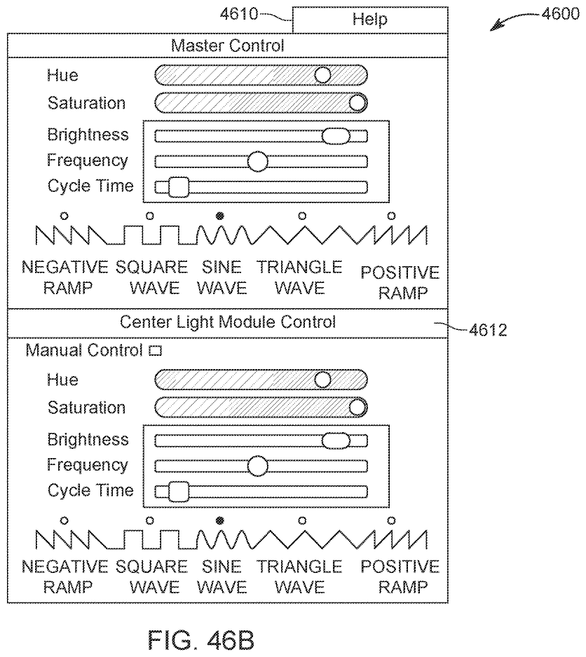

FIGS. 46A and 46B are a representation of a graphical user interface, designed to control specific mapped variables of a sensory stimulation feedback system according to an embodiment of the subject matter described herein. Here, a UI 4600 includes several panels including a main menu 4602, a control style panel 4604, a primary LED control panel 4606, an LED array control panel 4608, a Master Control panel 4601, and a Center light module control panel 4612.

FIG. 47 is a representation of a light and sound hybrid configuration of a sensory stimulation feedback system, including six full range speakers 4707 and one set of light emitters including the WS2813 layer ([subset B]) 4708 and six high powered RGBW LEDs (subset A) 4702, and a modular Halogen inner unit 4706 according to an embodiment of the subject matter described herein.

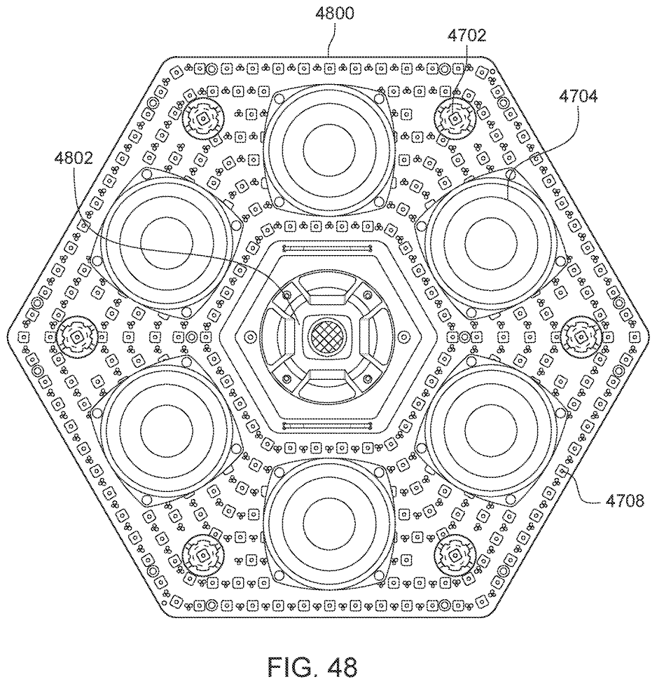

FIG. 48 is a representation of a light and sound hybrid configuration of a sensory stimulation feedback system, including six full range speakers and one set of light emitters including the WS2813 layer ([subset B]) and six high powered RGBW LEDs (subset A), and a modular speaker inner unit 4802 according to an embodiment of the subject matter described herein.

FIG. 49 is a representation of the internal electrical routing distribution hexagon structure of a sensory stimulation feedback system according to an embodiment of the subject matter described herein. Here, modules 4900 are associated with ingress lighting and include: UI module 4902, data interface module 4904, AC power interface module 4906, high intensity power interface module 4908, and WS2813 (small LED) power interface 4910.

As will be appreciated by one skilled in the art, aspects of the present invention may be embodied as a system, method or computer program product. Accordingly, aspects of the present invention may take the form of an entirely different hardware embodiment, an entirely different software embodiment (including firmware, resident software, micro-code, etc.) or an embodiment combining software and hardware aspects that may all generally be referred to herein as a “circuit,” “module” or “system.” Furthermore, aspects of the present invention may take the form of a computer program product embodied in one or more computer readable medium(s) having computer readable program code embodied thereon.

Any combination of one or more computer readable medium(s) may be utilized. The computer readable medium may be a computer readable signal medium or a computer readable storage medium (including, but not limited to, non-transitory computer readable storage media). A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium may be transmitted using any appropriate medium, including but not limited to wireless, wireline, optical fiber cable, RF, etc., or any suitable combination of the foregoing.

Computer program code for carrying out operations for aspects of the present invention may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter situation scenario, the remote computer may be connected to the user's computer through any type of network, including a local area network (LAN) or a wide area network (WAN), or the connection may be made to an external computer (for example, through the Internet using an Internet Service Provider).

Aspects of the present invention are described below with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

The flowchart and block diagrams in the Figures illustrate the architecture, functionality, and operation of possible implementations of systems, methods and computer program products according to various embodiments of the present invention. In this regard, each block in the flowchart or block diagrams may represent a module, segment, or portion of code, which comprises one or more executable instructions for implementing the specified logical function(s). It should also be noted, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

The terminology used herein is for the purpose of describing particular embodiments only and is not intended to be limiting of the invention. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A sensory stimulation device comprising:
an outer unit including a first set of emitters arranged substantially in a same plane and circuitry for supplying operating power to the first set of emitters;
an inner unit including a second set of emitters arranged substantially in a same plane and circuitry for supplying operating power to the second set of emitters;
a controller for operating the emitters based on an input that includes determining at least one of: a color, an intensity, a duration, a strobe effect, and a strobe frequency; and
a main body housing enclosing an inner volume when coupled to the outer unit,
wherein the outer unit is coupled to the main body housing,
wherein the inner unit is detachably coupled to the outer unit,
wherein the first and second sets of emitters emit electromagnetic radiation having a wavelength between about 1500 nm (infrared) and about 100 nm (ultraviolet).

2. The device of claim 1, wherein the input is received from an electroencephalogram (EEG) device associated with a user.

3. The device of claim 1, wherein operating the emitters includes activating a subset of the emitters in a pattern.

4. The device of claim 1, wherein the controller is further configured to adjust the pattern over time.

5. The device of claim 1, wherein the pattern includes continuously activating at least one emitter and discontinuously activating at least one emitter.

6. The device of claim 1, wherein the pattern includes at least one emitter emitting a first wavelength of light and at least another emitter emitting a second wavelength of light.

7. The device of claim 1, wherein the pattern includes dividing the plurality of emitters into at least a first group and a second group.

8. The device of claim 7, wherein the first group corresponds with the first set of emitters and wherein the second group corresponds with the second set of emitters.

9. The device of claim 7, wherein the first group includes emitters from both the first set of emitters and the second set of emitters and wherein the second group includes emitters from both the first set of emitters and the second set of emitters.

10. A method comprising:
receiving, by a controller associated with a sensory stimulation device including a first and a second set of emitters configured to emit electromagnetic radiation having a wavelength between about 1500 nm (infrared) and about 100 nm (ultraviolet), one or more inputs;
determining, by the controller, one or more control signals for the first and second sets of emitters based on the one or more inputs; and
supplying operating power and the control signals to the first set of emitters and the second set of emitters for controlling at least one of: a color, an intensity, a duration, a strobe effect, and a strobe frequency of the first and second sets of emitters.

11. The method of claim 10, wherein the input is received from an electroencephalogram (EEG) device associated with a user.

12. The method of claim 10, wherein operating the emitters includes activating a subset of the emitters in a pattern.

13. The method of claim 10, wherein the controller is further configured to adjust the pattern over time.

14. The method of claim 10, wherein the pattern includes continuously activating at least one emitter and discontinuously activating at least one emitter.

15. The method of claim 10, wherein the pattern includes at least one emitter emitting a first wavelength of light and at least another emitter emitting a second wavelength of light.

16. The method of claim 10, wherein the pattern includes dividing the plurality of emitters into at least a first group and a second group.

17. The method of claim 16, wherein the first group corresponds with the first set of emitters and wherein the second group corresponds with the second set of emitters.

18. The method of claim 16, wherein the first group includes emitters from both the first set of emitters and the second set of emitters and wherein the second group includes emitters from both the first set of emitters and the second set of emitters.

19. A non-transitory computer program product comprising:
a non-transitory computer readable storage medium having computer readable program code embodied therewith, the computer readable program code comprising computer readable program code configured for:
receiving, by a controller associated with a sensory stimulation device including a first and a second set of emitters configured to emit electromagnetic radiation having a wavelength between about 1500 nm (infrared) and about 100 nm (ultraviolet), one or more inputs;
determining, by the controller, one or more control signals for the first and second sets of emitters based on the one or more inputs; and
supplying operating power and the control signals to the first set of emitters and the second set of emitters for controlling at least one of: a color, an intensity, a duration, a strobe effect, and a strobe frequency of the first and second sets of emitters.

20. A sensory stimulation feedback system comprising:
an enclosure:
one or more interchangeable panels;
one or more sensors;
input devices;
access ports including an AC power input, unobstructed vents, and unobstructed microphone pathways;
wherein the enclosure physically connects to and provides communication interfaces with other devices, systems, and peripherals;
wherein the enclosure houses a modular system comprising:
a processing module configured to:
receive, store, and send data from internal, external, and
physically attached sources;
process data;
control other components located within, physically connected to, wirelessly interfaced with, and/or associated with the sensory stimulation feedback system;
operate emitters based on a previously created program, instructions from a hardware or software controller, or externally generated control signals;
a power distribution module configured to:
receive, convert, transmit, or route power to components of the system and charge internal and/or external batteries;
an outer unit comprising at least one of:
a first set of emitters arranged substantially in a same plane;
one or more subsets of emitters arranged substantially in multiple layered planes;
wherein the first set of emitters located on the outer unit emit electromagnetic radiation;
a modular inner unit comprising:
a second set of emitters that emit energy or matter (e.g., electromagnetic radiation having a wavelength different from the first set of emitters;
wherein the modular inner unit further comprises at least one of:
a second set of emitters arranged substantially in a same plane;
one or more subsets of emitters arranged substantially in multiple planes;
a single emitter;
one or more sensors;
one or more sensors in combination with one or more emitters;
wherein the modular inner unit can be attached to the outer unit and the processing module.
